(19) United States
(12) Patent Application Publication
(10) Pub. No.: US 2002/0034732 A1
(43) Pub. Date: Mar. 21, 2002

(54) COMPOSITIONS AND METHODS FOR DETERMINING ANTI-VIRAL DRUG SUSCEPTIBILITY AND RESISTANCE AND ANTI-VIRAL DRUG SCREENING

(76) Inventors: DANIEL J. CAPON, HILLSBOROUGH, CA (US); JEANNETTE M. WHITCOMB, SAN MATEO, CA (US); NEIL T. PARKIN, BELMONT, CA (US)

Correspondence Address: ALBERT WAI KAT CHAN, COOPER & DUNHAM, 1185 AVENUE OF THE AMERICAS, NEW YORK, NY, 10036

(21) Appl. No.: 09/126,559

(22) Filed: Jul. 30, 1998

Related U.S. Application Data
(60) Provisional application No. 60/054,257, filed on Jul. 30, 1997.

(51) Int. Cl. C12Q 1/70; C12Q 1/68; C12Q 1/18; C12P 13/14; C12N 1/20; C12N 15/00; C12N 15/09; C12N 15/63; C12N 15/70; C12N 15/74

(52) U.S. Cl. 435/5; 435/6; 435/32; 435/252.3; 435/320.1

(57) ABSTRACT

This invention provides a method for determining susceptibility for an HCV or HCMV anti-viral drug comprising: (a) introducing a resistance test vector comprising a patient-derived segment and an indicator gene into a host cell; (b) culturing the host cell from (a); (c) measuring expression of the indicator gene in a target host cell; and (d) comparing the expression of the indicator gene from (c) with the expression of the indicator gene measured when steps (a)-(c) are carried out in the absence of the anti-viral drug, wherein a test concentration of the anti-viral drug is present at steps (a)-(c); at steps (b)-(c); or at step (c). This invention also provides a method for determining HCV or HCMV anti-viral drug resistance in a patient comprising: (a) determining anti-viral drug susceptibility in the patient at a first time using the susceptibility test described above, wherein the patient-derived segment is obtained from the patient at about said time;(b) determining anti-viral drug susceptibility of the same patient at a later time; and (c) comparing the anti-viral drug susceptibilities determined in step (a) and (b) wherein a decrease in anti-viral drug susceptibility at the later time compared to the first time indicates development or progression of anti-viral drug resistance in the patient. This invention also provides a method for evaluating the biological effectiveness of a candidate HCV or HCMV anti-viral drug compound. Compositions including resistance test vectors comprising a patient-derived segment comprising a HCV or HCMV gene and an indicator gene and host cells transformed with the resistance test vectors are provided.

This application claims the benefit of U.S. Provisional Application No. 60/054,257, filed Jul. 30, 1997, the content of which is incorporated by reference into this application.

### BACKGROUND OF THE INVENTION

Viral Drug Resistance

The use of anti-viral compounds for chemotherapy and chemoprophylaxis of viral diseases is a relatively new development in the field of infectious diseases, particularly when compared with the more than 50 years of experience with antibacterial antibiotics. The design of anti-viral compounds is not straightforward because viruses present a number of unique problems. Viruses must replicate intracellularly and often employ host cell enzymes, macromolecules, and organelles for the synthesis of virus particles. Therefore, safe and effective anti-viral compounds must be able to discriminate with a high degree of efficiency between cellular and virus-specific functions. In addition, because of the nature of virus replication, evaluation of the in vitro sensitivity of virus isolates to anti-viral compounds must be carried out in a complex culture system consisting of living cells (e.g. tissue culture). The results from such assay systems vary widely according to the type of tissue culture cells which are employed and the conditions of assay.

Viral drug resistance is a substantial problem given the high rate of viral replication and mutation frequencies. Drug resistant mutants were first recognized for poxyiruses with thiosemicarbazone (Appleyard and Way (1966) Brit. J. Exptl. Pathol. 47, 144-51), for poliovirus with guanidine (Melnick et al. (1961) Science 134, 557), for influenza A virus with amantadine (Oxford et al. (1970) Nature 226, 82-83; Cochran et al. (1965) Ann. NY Acad Sci 130, 423-429) and for herpes simplex virus with iododeoxyuridine (Jawetz et al. (1970) Ann. NY Acad Sci 173, 282-291). Approximately 140 HIV drug resistance mutations to various anti-viral agents have been identified to date (Mellors et al. (1995) Intnl. Antiviral News, supplement and Condra, J. H. et al. (1996) J Virol. 70, 8270-8276). Approximately 20 human cytomegalovirus (HCMV) drug resistance mutations to various anti-viral agents have been identified to date (Biron (1996) Antiviral Chemotherapy, 4, 135-143).

The small and efficient genomes of viruses have lent themselves to the intensive investigation of the molecular genetics, structure and replicative cycles of most important human viral pathogens. As a consequence, the sites and mechanisms have been characterized for both the activity of and resistance to anti-viral drugs more precisely than have those for any other class of drugs. (Richman (1994) Trends Microbiol. 2, 401-407). The likelihood that resistant mutants will emerge is a function of at least four factors: 1) the viral mutation frequency; 2) the intrinsic mutability of the viral target site with respect to a specific anti-viral; 3) the selective pressure of the anti-viral drug; and, 4) the magnitude and rate of virus replication. with regard to the first factor, for single stranded RNA viruses, whose genome replication lacks a proofreading mechanism, the mutation frequencies are approximately 3×10⁻⁵ per base-pair per replicative cycle (Holland et al. (1992) Curr. Topics Microbiol Immunol. 176, 1-20; Mansky et al. (1995) J Virol. 69, 5087-94; Coffin (1995) Science 267, 483-489). Thus, a single 10 kilobase genome, like that of human immunodeficiency virus (HIV) or hepatitis C virus (HCV), would be expected to contain on average one mutation for every three progeny viral genomes. As to the second factor, the intrinsic mutability of the viral target site in response to a specific anti-viral agent can significantly affect the likelihood of resistant mutants. For example, zidovudine (AZT) selects for mutations in the reverse transcriptase of HIV more readily in vitro and in vivo than does the other approved thymidine analog d4T (stavudine).

One, perhaps inevitable consequence of the action of an anti-viral drug is that it confers sufficient selective pressure on virus replication to select for drug-resistant mutants (Herrmann et al. (1977) Ann NY Acad Sci 284, 632-7). With respect to the third factor, with increasing drug exposure, the selective pressure on the replicating virus population increases to promote the more rapid emergence of drug resistant mutants. For example, higher doses of AZT tend to select for drug resistant virus more rapidly than do lower doses (Richman et al. (1990) J. AIDS. 3, 743-6). This selective pressure for resistant mutants increases the likelihood of such mutants arising as long as significant levels of virus replication are sustained.

The fourth factor, the magnitude and rate of replication of the virus population, has major consequences on the likelihood of emergence of resistant mutants. Many virus infections are characterized by high levels of virus replication with high rates of virus turnover. (Perelson et al. (1996) Science, 271, 1582-1586; Nowak et al. (1996), PNAS 93, 4398-4402). This is especially true of chronic infections with HIV as well as hepatitis B and C viruses. The likelihood of emergence of AZT resistance increases in HIV-infected patients with diminishing CD4 lymphocyte counts which are associated with increasing levels of HIV replication (Ibid).

Higher levels of virus increase the probability of preexisting mutants. It has been shown that the emergence of a resistant population results from the survival and selective proliferation of a previously existing subpopulation that randomly emerges in the absence of selective pressure. All viruses have a baseline mutation rate. With calculations of approximately 10¹⁰ new virions being generated daily during HIV infection (Ho et al. (1995) Nature 373, 123-126), a mutation rate of 10⁻⁴ to 10⁻⁵ per nucleotide guarantees the preexistence of almost any single point mutation at any time point during HIV infection. Evidence is accumulating that drug resistant mutants do in fact exist in subpopulations of HIV infected individuals (Najera et al. (1994) AIDS Res Hum Retroviruses 10, 1479-88; Najera et al. (1995) J Virol. 69, 23-31; Havlir et al. (1996) J. Virol., 70, 7894-7899). The preexistence of drug resistant picornavirus mutants at a rate of approximately 10⁻⁵ is also well documented (Ahmad et al. (1987) Antiviral Res. 8, 27-39).

Hepatitis C Virus (HCV)

Hepatitis C virus (HCV) infection occurs throughout the world and, prior to its identification, represented the major cause of transfusion-associated hepatitis. The seroprevalence of anti-HCV in blood donors from around the world has been shown to vary between 0.02% and 1.23%. HCV is also a common cause of hepatitis in individuals exposed to blood products. There have been an estimated 150,000 new cases of HCV infection each year in the United States alone during the past decade (Alter 1993, Infect. Agents Dis. 2, 155-166; Houghton 1996, in Fields Virology, 3rd Edition, pp. 1035-1058).

The hepatitis C virus (HCV) is a member of the flaviviridae family of viruses, which are positive stranded, non-segmented, RNA viruses with a lipid envelope. Other members of the family are the pestiviruses (e.g. bovine viral diarrheal virus, or BVDV, and classical swine fever virus, or CSFV), and flaviviruses (e.g. yellow fever virus and Dengue virus). See Rice, 1996 in Fields Virology, 3rd Edition, pp. 931-959. Molecular dissection of HCV replication and hence understanding the functions of its encoded proteins, while greatly advanced by the isolation of the virus and sequencing of the viral genome, has been hampered by the lack of an efficient cell culture system for production of native or recombinant HCV from molecular clones. However, low-level replication has been observed in several cell lines infected with virus from HCV-infected humans or chimpanzees, or transfected with RNA derived from cDNA clones of HCV.

HCV replicates in infected cells in the cytoplasm, in close association with the endoplasmic reticulum (see FIG. 1). Incoming positive sense RNA is released and translation is initiated via an internal initiation mechanism (Wang et al. 1993, J. Virol. 67, 3338-3344; Tsukiyama-Kohara et al. 1992, J. Virol. 66, 1476-1483). Internal initiation is directed by a cis-acting RNA element at the 5′ end of the genome; some reports have suggested that full activity of this internal ribosome entry site, or IRES, is seen with the first 700 nucleotides, which spans the 5′ untranslated region (UTR) and the first 123 amino acids of the open reading frame (ORF) (Lu and Wimmer, PNAS 93, 1412, 1996). All of the protein products of HCV are produced by proteolytic cleavage of a large (3010-3030 amino acids, depending on the isolate) polyprotein, carried out by one of three proteases: the host signal peptidase, the viral self-cleaving metalloproteinase, NS2, or the viral serine protease NS3/4A (see FIG. 2). The combined action of these enzymes produces the structural proteins (C, E1 and E2) and non-structural (NS2, NS3, NS4A, NS4B, NS5A, and NS5B) proteins which are required for replication and packaging of viral genomic RNA. NS5B is the viral RNA-dependent RNA polymerase (RDRP) that is responsible for the conversion of the input genomic RNA into a negative stranded copy (complimentary RNA, or cRNA); the cRNA then serves as a template for transcription by NS5B of more positive sense genomic/messenger RNA.

Several institutions and laboratories are attempting to identify and develop anti-HCV drugs. Currently the only effective therapy against HCV is alpha-interferon, which can control the amount of virus in the liver and blood (viral load) in only a small proportion of infected patients (Houghton 1996, in Fields Virology, 3rd Edition, pp. 1035-1058). However, given the availability of the molecular structure of the HCV serine protease, NS3/4A (Love et al., 1996, Cell 87, 331-342; Kim et al. 1996, Cell 87, 343-355), and success using protease inhibitors in the treatment of HIV-1 infection, there should soon be alternatives available. In addition to HCV protease inhibitors, other inhibitors which might specifically interfere with HCV replication could target virus specific activities such as internal initiation directed by the IRES, RDRP activity encoded by NS5B, or RNA helicase activity encoded by NS3.

As a result of a high error rate of their RDRPs, RNA viruses are particularly able to adapt to many new growth conditions. Most polymerases in this class have an estimated error rate of 1 in 10,000 nucleotides copied. With a genome size of approximately 9.5 kb, at least one nucleotide position in the genome of HCV is likely to sustain a mutation every time the genome is copied. It is therefore likely for drug resistance to develop during chronic exposure to an anti-viral agent. As in the case of HIV, a rapid and convenient assay for drug resistant HCV would greatly improve the likelihood of successful antiviral therapy, given a selection of drugs and non-overlapping patterns of drug resistant genotypes. Resistance-associated mutations can sometimes be identified rapidly by growing the virus in cell culture in the presence of the drug, an approach used with considerable success for HIV-1. To date, however, a convenient cell culture system for HCV is lacking. It is therefore not possible to determine the precise nature of genetic changes which confer drug resistance in vitro. Thus, in the absence of a list of known resistance-associated mutations, the preferred resistance assay is one that relies on a phenotypic readout rather than a genotypic one.

Presently Available Drug Resistance Assays

There are no well established drug resistance assays for HCV currently available. However, several investigators have devised chimeric virus systems containing the HCV NS3 protease such that replication of the chimera, or expression of a reporter gene, is dependent on NS3 activity. These systems were devised in order to study various aspects of NS3 function and its cofactor, NS4A, and to serve as prototypes for cell-based drug screening assays.

Hirowatari et al (Anal. Biochem. 225:113, 1995) constructed an expression vector to synthesize an endoplasmic reticulum-tethered NS2-NS3-tax1 fusion protein (taxi is the transcriptional trans-activator from the HTLV-I retrovirus). Upon cleavage by NS3 (or NS2), the tax1 transactivator is released and migrates to the nucleus, where it acts to activate the expression of a reporter gene (CAT) controlled by the HTLV-1 LTR.

Hahm et al (Virology 226:318, 1996) constructed a chimeric poliovirus containing HCV NS3 upstream of the poliovirus polyprotein; replication of the chimera is dependent on NS3 protease activity, since liberation of the native N-terminus of the poliovirus polyprotein is essential for initiation of poliovirus replication. However, this chimera does not include the NS4A protein of HCV, which has been shown to modify the activity of NS3.

Filocamo et al (J. Virol. 71:1417, 1997) constructed a chimeric Sindbis virus containing HCV NS3/4A upstream of the Sindbis virus polyprotein; replication of the chimera is dependent on NS3 protease activity, since liberation of the native N-terminus of the Sindbis virus polyprotein is essential for initiation of Sindbis virus replication.

In vitro Expression Systems

Resistance test vectors rely on a cell culture system for transfection, replication, and expression of the indicator gene. Others have described systems for transfection of intact HCV RNA synthesized in vitro from cDNA constructs into Huh7 cells, and have demonstrated that replication can occur (Yoo et al. (1995) J. Virol. 69, 32-38). In addition, several cell lines have been identified which support HCV replication following infection with virus present in HCV-infected human or chimpanzee serum or plasma (Valli et al. (1997) Res. Virol. 148, 181-186; Shimizu et al. (1992) PNAS 89, 5477-5481; Shimizu et al. (1993) PNAS 90, 6037-6041; Shimizu and Yoshikura (1994) J. Virol. 68, 8406-8408; Shimizu et al.(1994) J. Virol. 68, 1494-1500; Mizutani et al. (1996) J. Virol. 70, 7219-7223). Currently these systems are limited to those in which replication is detected by sensitive methods such as RT/PCR, and are unlikely to allow for efficient production of an indicator gene (IG). Improved methods may soon become available as new cell lines and transfection methods are discovered. One example of a potential improvement would be to transfect the RNA as RNP complexes, prepared by in vitro transcription in the presence of purified NS5B, so that transcription can commence immediately upon uptake into the cells; this strategy has been applied to the negative-stranded RNA viruses such as influenza virus (Enami and Palese (1991) J. Virol. 65, 2711-2713, rabies virus (Schnell et al. (1994) EMBO J. 13, 4195-4203), and vesicular stomatitis virus (Lawson et al. (1995) PNAS 92, 4477-4481).

Since the genome length RNA of flaviviruses is infectious, HCV vectors may be in the form of a cDNA construct containing a promoter for the T7 RNA polymerase at the 5 end, and a T7 polymerase terminator sequence at the 3 end. Thus RNA can be synthesized in large quantities in vitro and transfected into cells. An alternative approach is to transfect DNA constructs, which contain a strong eucaryotic promoter (such as the CMV IE promoter), directly into cells. Potential advantages of transfection of RNA, rather than DNA, include the following: transfection of RNA circumvents potential cell-type specific restrictions of promoter activity; translation of recombinant protein usually occurs within minutes to hours following transfection of biologically active RNA, whereas translation following DNA transfection must be preceded by transcription and RNA processing events, which incurs delays of hours to days before maximal expression levels are reached. Representation of viral quasispecies is more straightforward when transfecting RNA since sufficient quantities of RNA can be synthesized from uncloned PCR products by including the sequence of a bacteriophage RNA polymerase in the 5′ PCR primer. This approach has been shown to preserve quasispecies diversity of poliovirus (Chumakov, J. Virol. 70:7331-7334, 1996). Generation of precise 5′ and 3′ termini on the RNA is more easily achieved by in vitro transcription, through the placement of the promoter sequence at the 5′ end and a restriction endonuclease recognition site at the 3′ end (used for DNA template linearization prior to transcription) relative to the viral sequences. However, the 3′ terminus may also be generated precisely via the placement of a self-cleaving RNA ribozyme sequence, present in a cDNA construct (e.g. see Chowrira et al. (1994), J. Biol. Chem. 269, 25856-25864.).

A third transfection strategy, which possesses some of the advantages of RNA transfection, is DNA transfection of constructs containing a T7 RNA polymerase promoter at the 5′ end, and a T7 RNA polymerase terminator at the 3′ end, into cells which express the T7 RNA polymerase. Expression of the polymerase may be achieved by various means, perhaps the most efficient of these being the infection of the transfected cells with a recombinant T7 polymerase/vaccinia virus (Fuerst et al. (1986) PNAS 83, 8122-8126.)

Human Cytomegalovirus (HCMV)

Human Cytomegalovirus (HCMV) is endemic throughout the world and infection rates appear to be relatively constant throughout the year rather than seasonal. Humans are the only known reservoir for HCMV and natural transmission occurs by direct or indirect person-to-person contact. Between 0.2% and 2.2% of infants born in the United States are infected in utero. Another 8% to 60% become infected during the first six months of life as a result of infection acquired during birth or following breast feeding. Because of the high incidence of reactivation of HCMV infection in the breast, breast milk transmission could represent the most common mode of HCMV transmission worldwide. In most developed countries, 40% to 80% of children are infected before puberty. In other areas of the world, 90% to 100% of the population become infected during childhood.

Human cytomegalovirus (HCMV) is a member of the herpesvirus family. A typical herpes virion consists of a core containing a linear double-stranded DNA and icosadeltahedral capsid approx. 100-110 nm in diameter containing 162 capsomeres with a hole running down the long axis, an amorphous “tegument” that surrounds the core and an envelope containing viral glycoprotein spikes on its surface. Virion sizes range from 120-300 nm due to differences in the thickness of the tegument layer. There are three subgroups of herpesviruses:

1. Alphaherpesvirinae: HSV, VZV. variable host range, relatively short reproductive cycle, rapid spread in culture, efficient destruction of infected cells, capacity to establish latent infections in sensory ganglia.

2. Betaherpesvirinae: HCMV. Restricted host range, long reproductive cycle, slow progression of infection in culture. Infected cells become enlarged and carrier cells are readily established. Virus can be maintained in latent form in secretory glands, lymphoreticular cells, kidneys and other tissues.

3. Gammaherpesvirinae: EBV. experimental host range extremely narrow, replicate in lymphoblastoid cells and cause lytic infections in some types of epithelial and fibroblastoid cells.

There are 8 known human herpesviruses: Human herpesvirus 1 (Herpes simplex virus 1, HSV-1), Human herpesvirus 2 (Herpes simplex virus 2, HSV-2), Human herpesvirus 3 (Varicella-zoster virus, VZV), Human herpesvirus 4 (Epstein-Barr virus, EBV), Human herpesvirus 5 (Human cytomegalovirus), Human herpesvirus 6, Human herpesvirus 7, and Human herpesvirus 8. The genomes of herpes viruses consist of a linear double-stranded (ds) DNA in the virion which circularizes and concatamerizes upon release from the virus capsid in the nucleus of infected cells (See FIG. 10). The genomes of herpesviruses range in size from 120 to 230 kilobase pairs (kbp). The genomes are polymorphic in size (up to 10 kbp differences) within an individual population of virus. This variation is due to the presence of terminal and internal reiterated sequences. Herpes viruses can be classified into six groups, A through F, based on their overall genome organization. HSV and HCMV fall into group E, in which sequences from both termini are repeated in an inverted orientation and juxtaposed internally, dividing the genomes into two components, L (long) and S (short), each of which consists of unique sequences, U_{L} and U_{S}, flanked by inverted repeats (FIG. 10). In these viruses both components can invert relative to each other and DNA extracted form virions consists of four equimolar populations differing in the relative orientation of the two components (See FIG. 11).

HCMV is a betaherpesvirus and is unique among the betaherpesvirinae in that it falls into the class E genome type. The genome of HCMV is approximately 230 kbp in length and has been completely sequenced (EMBL Seq database accession # X17403) and contain 208 predicted open reading frames (ORFs) of greater than 100 amino acids in length (Mocarski, E. S. (1996) Ch.76 In Fields Virology, Third Edition edited by B. N. Fields, D. M. Knipe, P. M. Howley, etal. 2447-2492; Chee et al. (1990) Curr top Microbiol Immunol 154:125-170). ORFs are designated by their location within the unique and repeated regions of the viral genome (TRL, UL, IRL, IRS and US) and are numbered sequentially.

In a naturally occurring population of virus the genome exists in 4 isomers (See FIG. 11). In HCMV, as in HSV, the L-S junction can be deleted, thereby freezing the genome in one of four isomers without dramatically affecting the ability of the virus to grow in cultured cells.

The HCMV genome contains terminal repeat sequences “a” and “a′” present in a variable number in direct orientation at both ends of the linear genome. A variable number of “a” repeats are also present in an inverted orientation at the L-S junction. The number of “a” sequences in these locations ranges from 1-10 with 1 predominating. The size of “a” in HCMV ranges from 700-900 bp. The “a” sequence carries the cleavage and packaging signal. The packaging signals are two highly conserved short sequence elements located within “a” designated pac-1 and pac-2. A 220-bp fragment that carries both the pac-1 and pac-2 elements is sufficient to convey sites for cleavage/packaging as well as inversion on a recombinant CMV construct. The termini of the linear genome are generated by a cleavage event that leaves a single 3′ overhanging nucleotide at either end of the genome. The genome is further characterized by large inverted repeats called “b” and “b′” (or TRL and IRL) and “c” and “c” (or IRS and TRS) that flank unique sequences U_{L} and U_{S} that make up the L and S components of the genome (See FIG. 10)

The HCMV replication cycle is relatively slow compared to other herpesviruses. Viral replication involves the ordered expression of consecutive sets of viral genes. These sets are expressed at different times after infection and include the a (immediate early), β1 and β2 (delayed early), and γ1 and γ2 (late) sets based on the time after infection that their transcripts accumulate. DNA replication, genome maturation and virion morphogenesis are coordinated through the temporal regulation of the appropriate gene products required for each step. Expression of a gene products is rapid. Late gene expression is delayed for 24-36 hours. Progeny virions begin to accumulate 48 hours post-infection and reach maximal levels at 72-96 hours. In permissive fibroblasts, DNA replication can be detected as early as 14-16 hours post-infection. HCMV stimulates host DNA, RNA and protein synthesis. HCMV replicates more rapidly in actively dividing cells and HCMV replication is inhibited by pretreating cells with agents that reduce host cell metabolism. The HCMV genome circularizes soon after infection. Circles give rise to concatamers and genomic inversion occurs within concatameric forms of the DNA. The majority of replicating DNA is larger than unit length and lacks terminal fragments based on southern blot analysis.

Targets for Drug Resistance

The drugs currently used to treat HCMV (ganciclovir (GCV), foscarnet, cidofovir) are known to select for mutations in two viral genes, the UL97 phosphotransferase and the UL54 viral DNA polymerase.

UL97: phosphotransferase 707 amino acids (aa) (2121 bp) Mutations associated with GCV resistance include aa#: 460, 520, 590, 591, 592, 593, 594, 595, 596, 600, 603, 607, 659, 665. The phosphotransferase protein has two functional domains, 1) the amino terminal 300 aa code for a regulatory domain and 2) the carboxy terminal 400 aa define the catalytic domain. All known drug-resistance mutations are found in the catalytic domain (approx 1.2 kb of sequence). In HSV the thymidine kinase gene product (TK) is responsible for the phosphorylation of GCV in cells and resistance to GCV in HSV is associated with mutations in the thymidine kinase gene. HCMV has no homolog to the HSV thymidine kinase gene. The gene homologous to UL97 in HSV (UL13) is a protein kinase.

UL54: viral DNA polymerase, 1242 a.a. (3726 bp). Mutations in this gene can result in resistance to GCV and other nucleoside analogs (including cidofovir) as well as foscarnet. Mutations associated with foscarnet resistance include aa #: 700 and 715. Mutations associated with GCV resistance include aa#: 301, 412, 501, 503, and 987. The mature protein has four recognized domains: 1) a 5′-3′ exoRNase H, a 3′-5′ exonuclease, a proposed catalytic domain and an accessory protein binding domain.

New therapies in development include agents targeted to the CMV protease (UL80) and the DNA maturational enzyme (“terminase”).

GCV-resistant HCMV has been recovered from the central nervous system (CNS) of patients with HCMV-associated neurologic disease who had received long-term GCV maintenance therapy. Resistant strains of HCMV may be selected and preferentially located in the CNS. It is frequently not possible to culture virus from the cerebral spinal fluid (CSF) but it is possible to amplify HCMV DNA using PCR.

Primary isolates of CMV may replicate slowly. In addition, there is a marked delay in the growth rate of some of the drug resistant clinical isolates. In a mixed virus population, a resistant virus population could be masked by a sensitive one. Thus assay results that depend on the growth of virus could be unreliable.

Most assays for viral culture use blood or urine, because they are easy to obtain. However, the virus from these compartments may not represent the virus in specific tissues where disease is occurring (especially vitreous fluid and Csf). Although there are a few amino acid residues that are modified relatively frequently among drug-resistant strains of herpesviruses recovered from patients, the broad distribution of mutations in the majority of strains makes rapid genetic screening methods impractical. Importantly, since the drug-susceptibility phenotypes resulting from individual genetic changes are complex and variable, a biological test for anti-viral susceptibility of HCMV would be more informative.

Presently Available Viral Resistance Assays

The definition of viral drug susceptibility is generally understood to be the concentration of the anti-viral agent at which a given percentage of viral replication is inhibited (e.g. the IC₅₀ or an anti-viral agent is the concentration at which 50% of virus replication is inhibited). Thus, a decrease in viral drug susceptibility is the hallmark that an anti-viral has selected for mutant virus that is resistant to that anti-viral drug. Viral drug resistance is generally defined as a decrease in viral drug susceptibility in a given patient over time. In the clinical context, viral drug resistance is evidenced by the anti-viral drug being less effective or no longer being clinically effective in a patient.

Several types of assays are available to detect and measure antiviral drug susceptibility of HCMV. The two most commonly used methods are a plaque reduction assay and a DNA hybridization assay. At present the plaque reduction assay is considered the standard. Both assays require HCMV isolation and passage in cell culture. Generally, it takes four to six weeks to obtain the results from the assays.

Plaque reduction assays with increased sensitivity can now be performed directly on clinical specimens, including blood, urine, bronchoalveolar lavage, and cerebrospinal fluid. Two assays which are modified from the standard plaque reduction assay detect either the CMV immediate-early antigen or late antigen. The procedure is essentially the same as the standard plaque reduction assay except that the virus is tested directly without prior passage and the incubation time is reduced to ninety-six hours (Gerna et al. (1995) J. Clin. Microbiol. 33, 738-741). The limitation of these assays is that they can only be performed in patients with high level of viremia. Virus culture remains an essential step in the detection of drug resistant isolates.

An alternative approach is the detection of specific viral DNA mutations related to drug resistance. In this assay, PCR primers are used to amplify viral DNA and restriction sites present in mutant viral DNA but not wildtype DNA are used to determine the genotype of the viral DNA. It is suggested that the analysis of two PCR products with a total of three or four restriction digests is adequate to detect 78-83% of UL97 (certain mutations of UL97 which codes for a phosphotransferase, result in resistance to ganciclovir) mutants resistant to ganciclovir (Chou et al. (1995) J. Infect. Dis. 172, 239-242.). The main limitation of this assay is that infrequent or new resistance mutations are not identified. Also, DNA polymerase mutations (UL54) which are indicative of high-level ganciclovir resistance and a high probability of multidrug resistance are not detected.

It is an object of this invention to provide a drug susceptibility and resistance test capable of showing whether a viral population in a patient is resistant to a given prescribed drug. Another object of this invention is to provide a test that will enable the physician to substitute one or more drugs in a therapeutic regimen for a patient that has become resistant to a given drug or drugs after a previous course of therapy. Yet another object of this invention is to provide a test that will enable selection of an effective drug regimen for the treatment of virus infections. Yet another object of this invention is to provide a safe, standardized, affordable, rapid, precise and reliable assay of drug susceptibility and resistance for clinical and research application. Still another object of this invention is to provide a test and methods for evaluating the biological effectiveness of candidate drug compounds which act on specific viral genes and/or viral proteins particularly with respect to viral drug resistance and cross resistance. It is also an object of this invention to provide the means and compositions for evaluating viral drug resistance and susceptibility. This and other objects of this invention will be apparent from the specification as a whole.

### BRIEF DESCRIPTION OF THE FIGURES

FIG. 1: HCV Replication

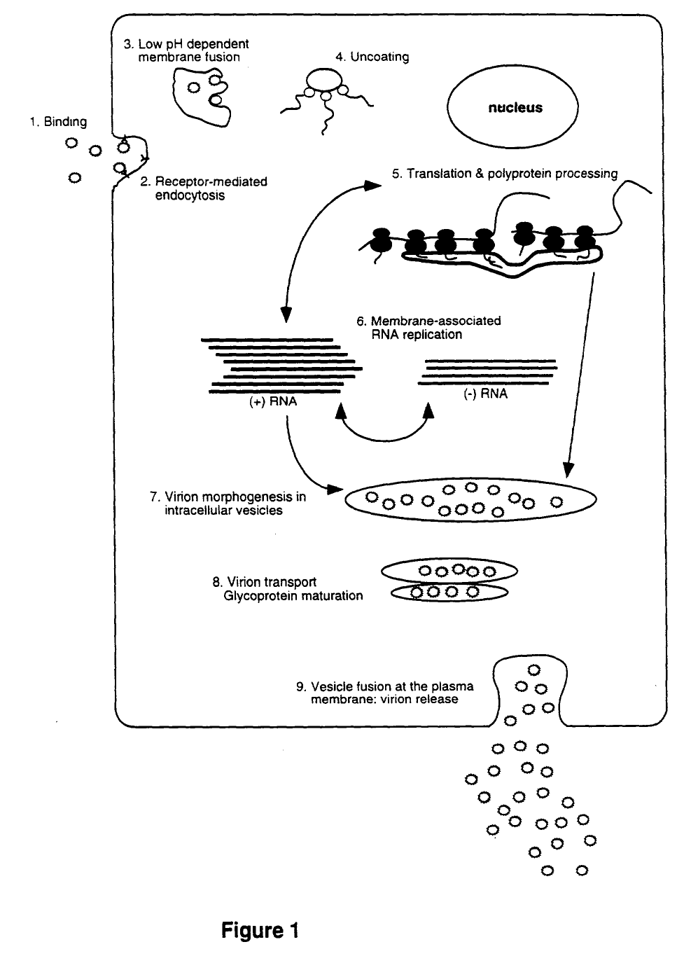

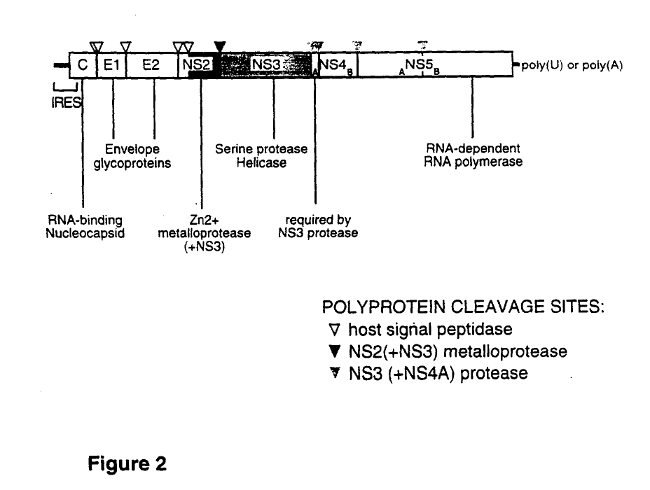

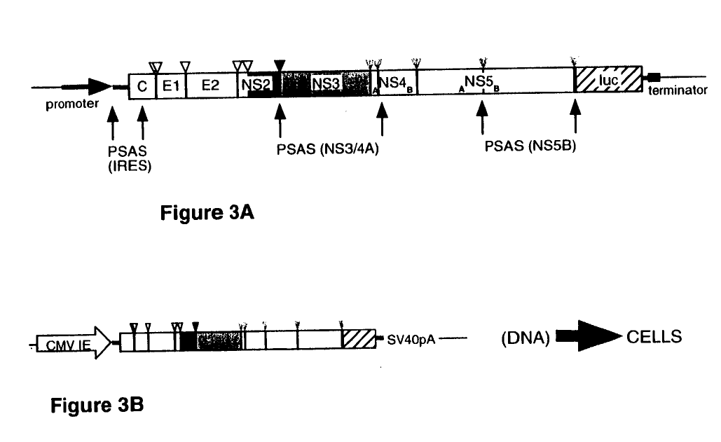

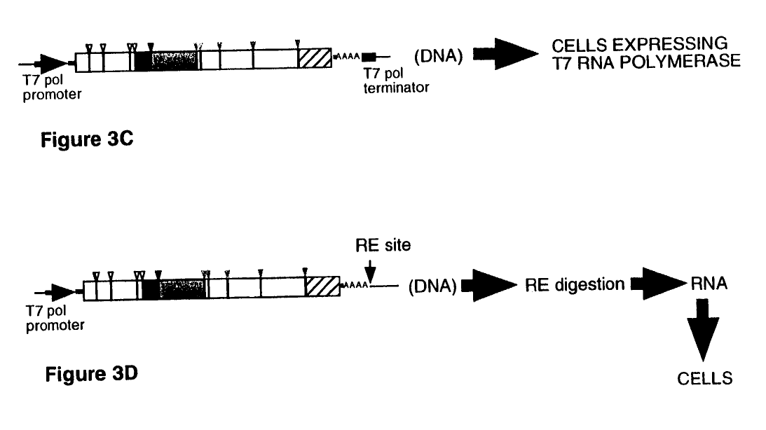

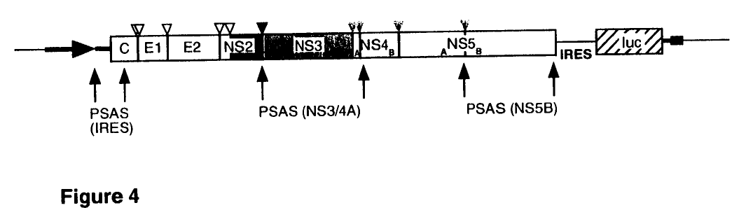

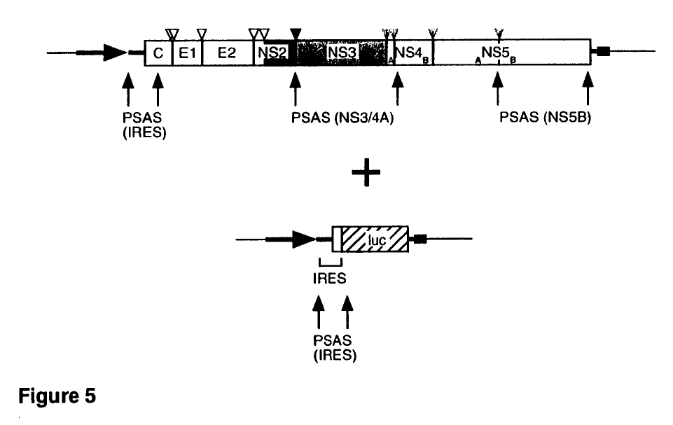

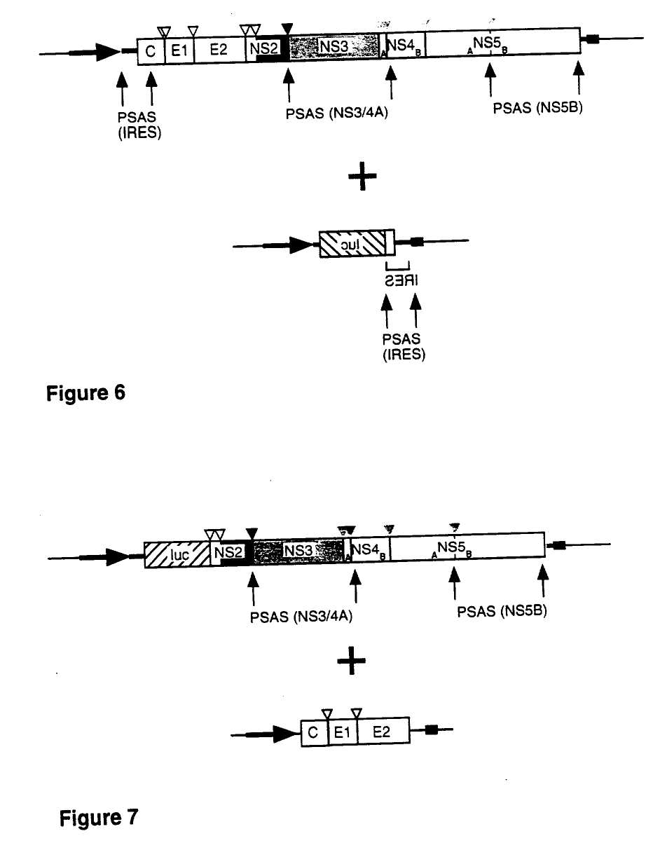

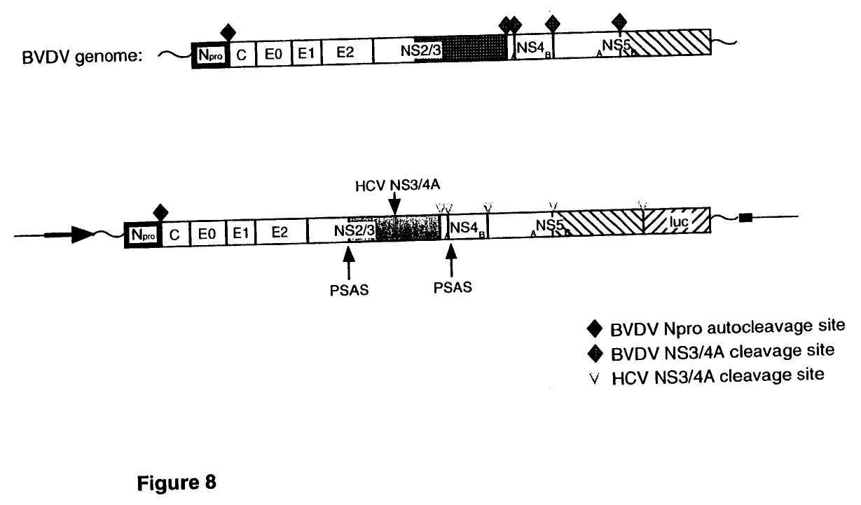

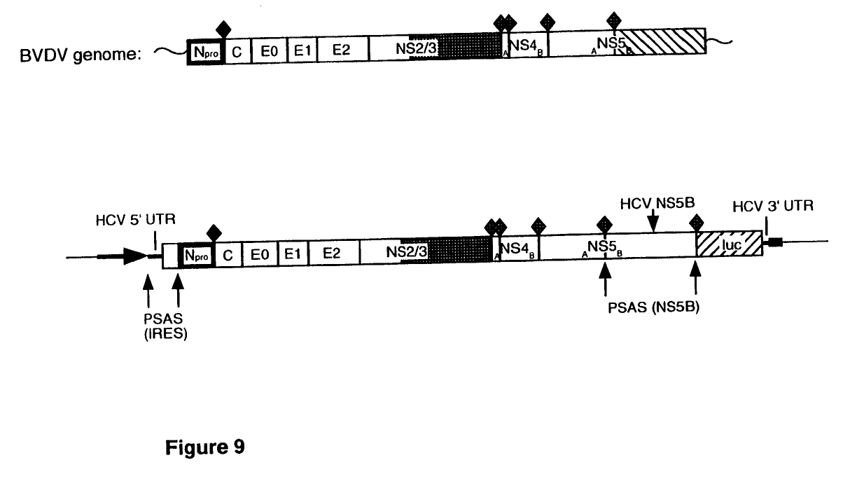

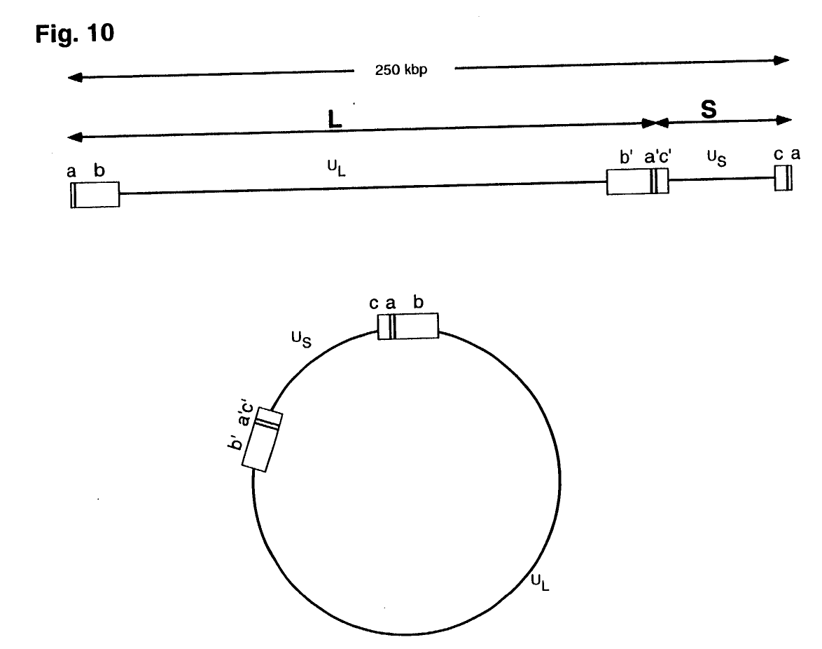

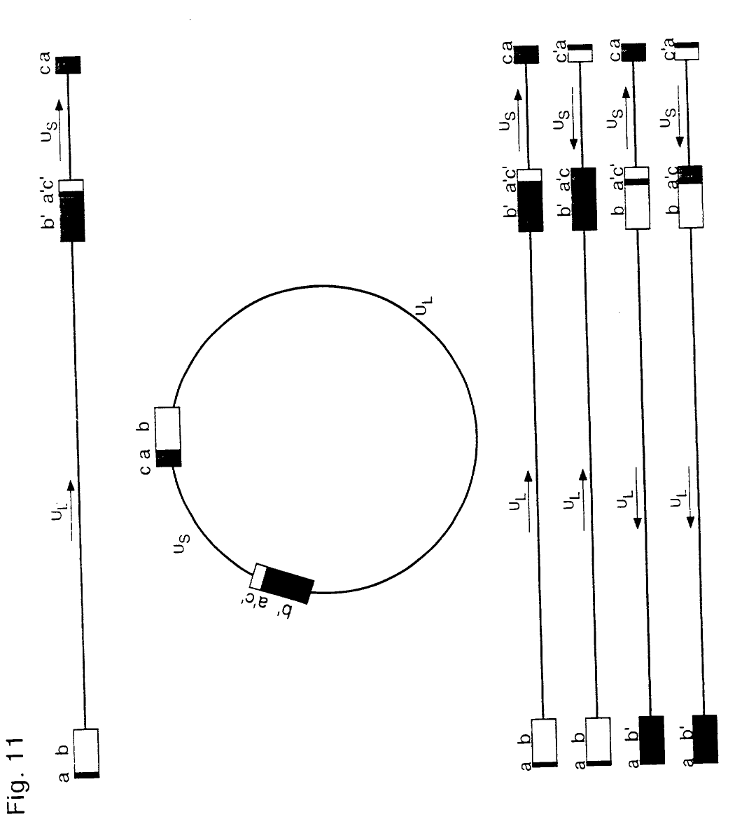

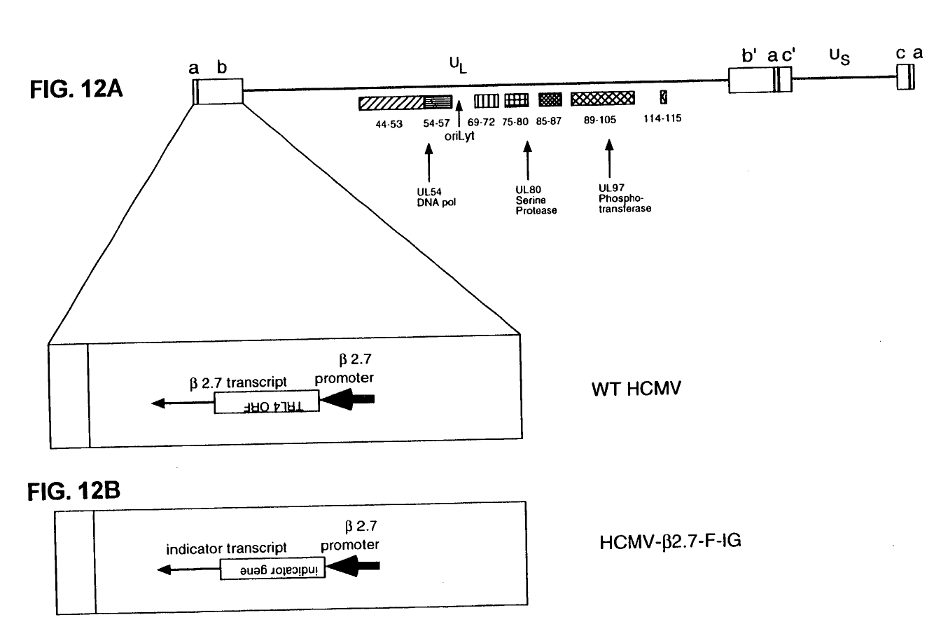

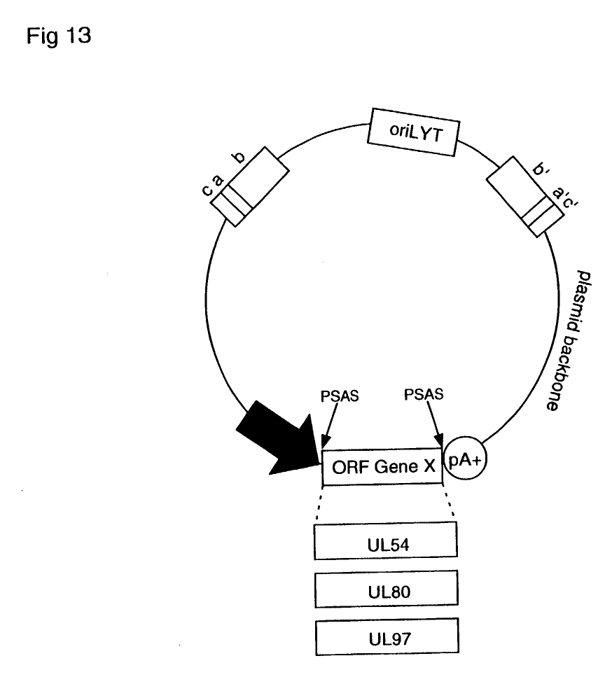

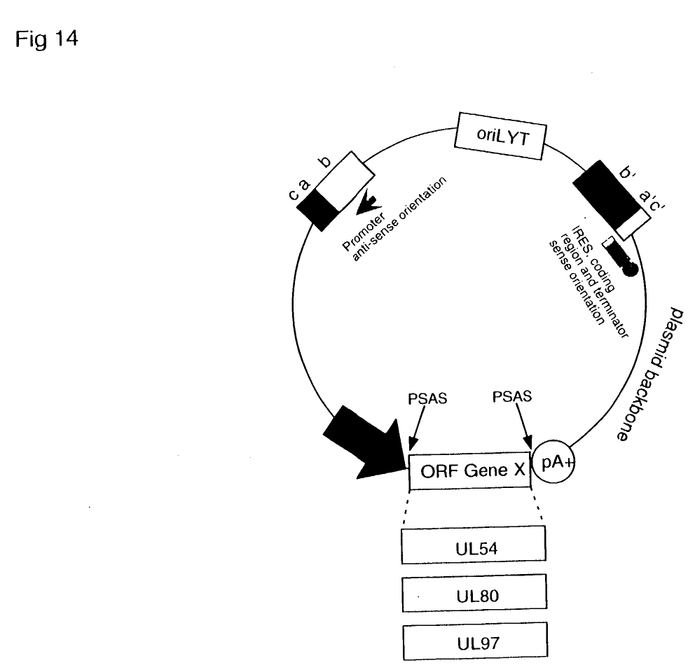

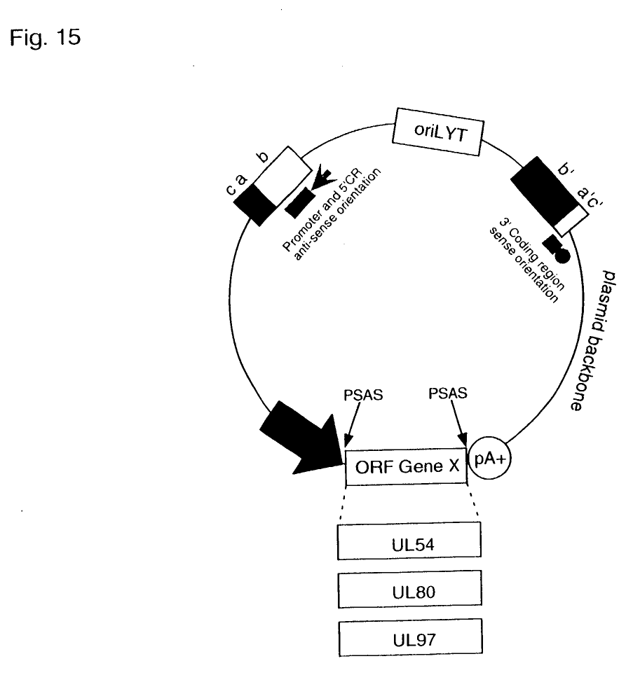

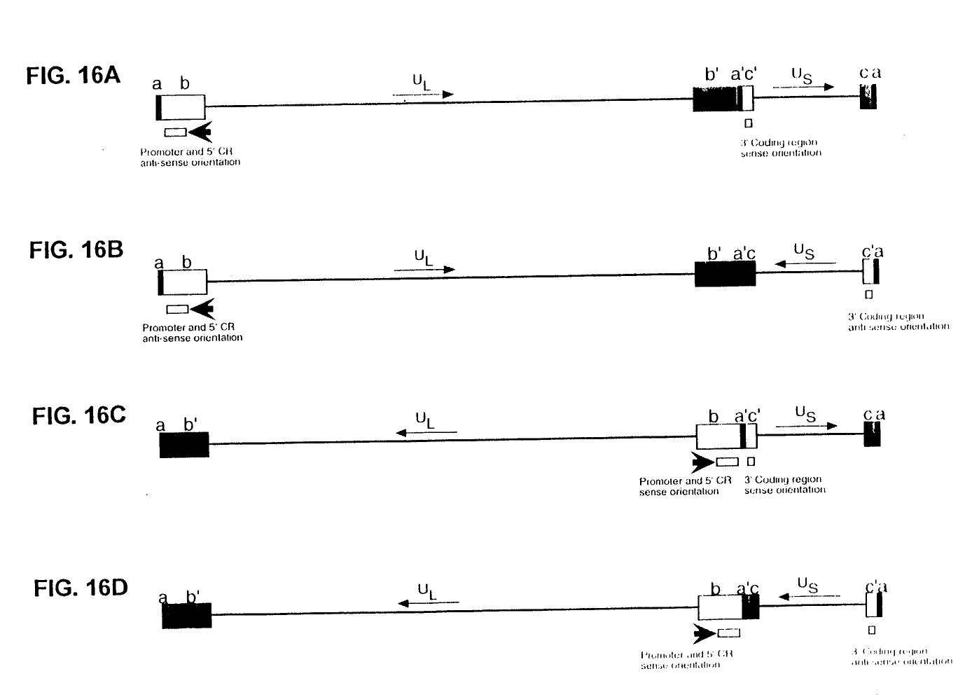

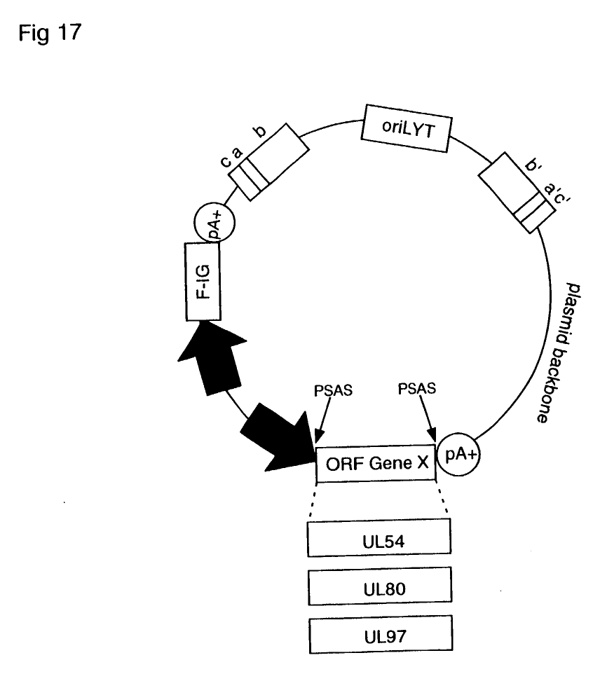

Schematic drawing of the replication cycle of HCV. Virions bind to the cell surface, via a specific interaction between a viral surface glycoprotein and a cell surface receptor (1). Following receptor-mediated endocytosis (2) and low pH dependent membrane fusion (3), the nucleocapsid core is released into the cytoplasm (4). Virion RNA is translated in close association with the endoplasmic reticulum, and the polyprotein is processed by specific endoproteolytic cleavages mediated by host signal peptidase in the ER, or one of two viral proteases (5). After enough of the non-structural proteins have been produced, the viral RNA is replicated through a negative strand intermediate, to generate more positive sense RNA for translation and packaging into new virions (6). Structural proteins and RNA assemble to form new viral particles which bud into the ER (7) and are secreted via the cellular pathway (8, 9) to release the progeny virions.

FIG. 2: HCV Genome Structure

Schematic diagram of the ˜9.5 kb HCV RNA. The order of the individual HCV proteins is indicated in the HCV polyprotein, with putative functions associated indicated below. Cleavage sites for proteolytic processing are indicated by triangles (open triangles for host signal peptidase, black triangle for NS2/3, and grey triangles for NS3/4A). The internal ribosome entry site (IRES) is located at the 51 end of the RNA and comprises the entire untranslated region (UTR) and some sequences at the beginning of the C ORF. The 3′ end of the RNA contains either a poly(A) or poly(U) tail, depending on the type of HCV.

FIG. 3: Resistance Test Vectors (Luciferase Fusion Protein).

A. Diagrammatic representation of the resistance test vector (pXHCV-luc, where X is either CMV or T7), with patient sequence acceptor sites for transfer of patient derived segments indicated by arrows below the polyprotein (PSAS). The promoter and terminator sequences are indicated generically in this figure as well as in subsequent figures, as several different types of regulatory elements may be used (as described below). The luciferase reporter gene is expressed as a fusion protein with the HCV polyprotein and then cleaved off by the action of NS3/4A.

B. Method for transfection using DNA transfection of a resistance test vector (pCMVHCV-luc) containing the CMV IE promoter and SV40 polyadenylation signal. The RNA is transcribed in the nucleus of transfected cells by cellular RNA polymerases, then transported to the cytoplasm where translation and replication can occur.

C. Method for transfection using DNA transfection of a resistance test vector (pT7HCV-luc1) containing the T7 RNA polymerase promoter and T7 RNA polymerase terminator. The DNA is transfected into cells expressing T7 RNA polymerase (for example, after infection with recombinant vaccinia virus or by co-transfection with a T7 RNA polymerase expression plasmid); RNA is transcribed in the cytoplasm by T7 polymerase.

D. Method for transfection using RNA transfection of RNA derived from a resistance test vector (pT7HCV-luc2) containing the T7 RNA polymerase promoter and a restriction site placed at the 3′ end for linearization of the DNA prior to transcription in vitro. The synthetic RNA is then transfected directly into cells and translation and replication can occur.

FIG. 4: Resistance Test Vectors (Bicistronic Luciferase Expression).

Structure of the resistance test vector (pXHCV-IRESluc) containing an IRES element for luciferase translation. The IRES may be the native HCV IRES, or derived from other viruses which use such elements for internal initiation of their mRNAs. Expression of luciferase occurs by internal initiation of translation from the bicistronic RNA in the cytoplasm of transfected cells.

FIG. 5: Resistance Test Vectors (Positive Sense Minigenomes).

Diagrammatic representation of the resistance test vectors (pXHCV and pXIRESluc) comprising a positive sense luciferase RNA minigenome. The two constructs are co-transfected into cells; HCV non-structural proteins expressed from PXHCV act on both RNAs to replicate and package them. The replicated RNA are packaged into progeny virions which can then be used for infection of fresh target cells; the target cells are also infected with HCV or transfected with pXHCV, and the luciferase minigenome is expressed.

FIG. 6: Resistance Test Vectors (Negative Sense Minigenomes).

Diagrammatic representation of the resistance test vector (pXHCV-ASIRESluc) comprising a negative sense RNA minigenome. The two constructs are co-transfected into cells; HCV non-structural proteins expressed from pXHCV act on both RNAs, leading to their replication. The replicated RNA are packaged into progeny virions which can then be used for infection of fresh target cells; the target cells are also infected with HCV or transfected with pXHCV, and the luciferase minigenome (now positive sense RNA) is expressed.

FIG. 7: Resistance Test Vectors (Defective Genome).

Diagrammatic representation of the resistance test vectors (pXluc-NSHCV and PXSHCV) expressing defective genomic RNAs. The two constructs are co-transfected into cells; non-structural proteins expressed from pXluc-NSHCV act to replicate the luc-NSHCV RNA; the newly replicated RNA is packaged into virions using structural proteins (C, E1 and E2) from PXSHCV. The progeny virions are then used to infected new cells.

FIG. 8: Resistance Test Vectors (BVDV NS3/4A Chimeras, luc Fusion Protein)

A diagrammatic representation of the genome of BVDV is shown at the top. HCV protease cleavage sites are indicated by grey triangles, and BVDV protease cleavage sites are represented by crosshatched diamonds (signal peptidase and NS2/3 protease cleavage sites are not shown). The resistance test vector pXBVDV(HCVNS3)luc contains the BVDV structural protein genes, BVDV NS2, HCV NS3/4A protease, and BVDV NS4B and NS5; the cleavage sites in the nonstructural protein region are altered so that they are recognized by the HCV NS3/4A protease. The luciferase reporter gene is expressed as a fusion with the chimeric polyprotein, and released by cleavage by HCV NS3/4A.

FIG. 9: Resistance Test Vectors (BVDV NS5B Chimeras, luc Fusion Protein).

The resistance test vector pXBVDV(HCVNS5B)luc comprising the BVDV structural protein genes, BVDV NS2, NS3/4A protease, NS4B and NS5A, and HCV NS5B; the cis-acting regulatory elements recognized by the NS5B polymerase, located in the 3′ UTR and 5′ UTR and amino terminal region of the C ORF, are derived from HCV. The luciferase reporter gene is expressed as a fusion with the chimeric polyprotein, and released by cleavage by BVDV NS3/4A.

FIG. 10:

A. Diagrammatic representation of the HCMV genome. The genome has terminal direct repeats designated as “a” which exist in 1-10 copies per genome. The “a” sequences are also present in an inverted orientation at the L-S junction (a′). Inverted repeats “b” and “c” are designated as blocks, “b′” and “c′” are used to designate the “b” and “c” repeats in the anti-sense orientation. U_{L} and U_{S} designate the unique regions of the L and S components of the genome. Blocks of ORFs are shown below the genome. Three genes that code for targets of anti-viral drugs, UL54, UL80 and UL97 are indicated by arrows. OriLYT refers to the HCMV lytic origin of replication.

B. Circularization of a monomeric HCMV genome following infection

FIG. 11:

A. Diagrammatic representation of the HCMV genome.

B. Circularization of the HCMV genome following infection.

C. Four isomers of HCMV genome following inversion of the genome during replication. Arrows under the U_{L} and U_{S} segments emphasize the inversion of the L and S segments of the genome relative to each other.

FIG. 12:

Diagrammatic representation of the HCMV genome. The β_{2.7} transcript present in the “b” region of the genome is shown as it exists in the wild type HCMV (A) and as it is modified in the HCMV-β_{2.7}-F-IG (B)

FIG. 13:

Diagrammatic representation of the amplicon plasmid lacking a functional indicator gene. ORF Gene X designates an anti-viral target (e.g. UL54, UL80, UL97) The large black arrow represents a promoter and the circle (pA+) indicates a polyadenylation signal. The promoter and polyadenylation signal can be derived from the HCMV genome and appropriate to the viral gene/drug target (gene X) or may be exogenous regulatory elements as described in the text. PSAS indicates patient sequence acceptor sites.

FIG. 14:

Diagrammatic representation of the amplicon plasmid comprising a non-functional indicator gene which includes a permuted promoter. ORF Gene X designates an anti-viral target (e.g. UL54, UL80, UL97) The large black arrow represents a promoter and the round circle (pA+) indicates a polyadenylation signal. The promoter and polyadenylation signal can be derived from the HCMV genome and appropriate to the viral gene/drug target (gene X) or may be exogenous regulatory elements as described in the text. PSAS indicates patient sequence acceptor sites. This amplicon contains the permuted promoter cassettes as described in the text.

FIG. 15:

Diagrammatic representation of the amplicon plasmid comprising a non-functional indicator gene with a permuted coding region. ORF Gene X designates an anti-viral target (e.g. UL54, UL80, UL97) The large black arrow represents a promoter and the circle (pA+) indicates a polyadenylation signal. The promoter and polyadenylation signal can be derived from the HCMV genome and appropriate to the viral gene/drug target (gene X) or may be exogenous regulatory elements as described in the text. PSAS indicates patient sequence acceptor sites. This amplicon contains the permuted coding region cassettes as described in the text.

FIG. 16:

Diagrammatic representation of the four isomers of the HCMV genome present after viral replication and the relative position of the permuted coding region cassettes after rearrangement. Note that in panel C the 2 halves of the cassette are now in the proper orientation to direct expression of the reporter gene. The arrangement shown in panel B will also result in an appropriate juxtaposition of the 2 halves of the cassette following concatamerization of the rearranged genomes.

FIG. 17:

Diagrammatic representation of amplicon plasmid comprising a functional indicator gene. ORF Gene X designates an anti-viral target (e.g. UL54, UL80, UL97) The large black arrow represents a promoter and the circle (pA+) indicates a polyadenylation signal. The promoter and polyadenylation signal can be derived from the HCMV genome and appropriate to the viral gene/drug target (gene X) or may be exogenous regulatory elements as described in the text. PSAS indicates patient sequence acceptor sites.

This amplicon contains the functional indicator gene cassette as described in the text.

### DETAILED DESCRIPTION OF THE INVENTION

In order that the invention described herein may be more fully understood, the following description is set forth.

The following flow chart illustrates certain of the various vectors and host cells which may be used in this invention. It is not intended to be all inclusive.

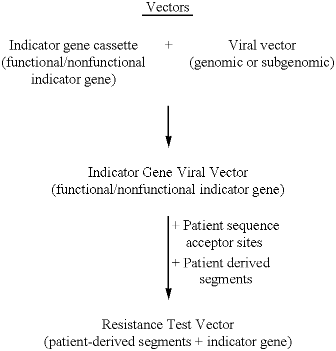

Host Cells

Packaging Host Cell—transfected with packaging expression vectors

Resistance Test Vector Host Cell—a packaging host cell transfected with a resistance test vector

Target Host Cell—a host cell to be infected by a resistance test vector viral particle produced by the resistance test vector host cell. The component of the resistance test vector system that contains the indicator gene can be delivered to the target host cell at the time of infection or may be stably integrated into the target host cell chromosomal DNA.

Resistance Test Vector

“Resistance test vector” means one or more vectors which taken together contain DNA or RNA comprising a patient-derived segment and an indicator gene. In the case where the resistance test vector comprises more than one vector the patient-derived segment may be contained in one vector and the indicator gene in a different vector. Such a resistance test vector comprising more than one vector is referred to herein as a resistance test vector system for purposes of clarity but is nevertheless understood to be a resistance test vector. The DNA or RNA of a resistance test vector may thus be contained in one or more DNA or RNA molecules. In one embodiment, the resistance test vector is made by insertion of a patient-derived segment into an indicator gene viral vector. In another embodiment, the resistance test vector is made by insertion of a patient-derived segment into a packaging vector while the indicator gene is contained in a second vector, for example an indicator gene viral vector. As used herein, “patient-derived segment” refers to one or more viral segments obtained directly from a patient using various means, for example, molecular cloning or polymerase chain reaction (PCR) amplification of a population of patient-derived segments using viral DNA or complementary DNA (cDNA) prepared from viral RNA, present in the cells (e.g. peripheral blood mononuclear cells, PBMC), serum or other bodily fluids of infected patients. When a viral segment is “obtained directly” from a patient it is obtained without passage of the virus through culture, or if the virus is cultured, then by a minimum number of passages to essentially eliminate the selection of mutations in culture.

The term “viral segment” refers to any functional viral sequence or viral gene encoding a gene product (e.g., a protein) that is the target of an anti-viral drug. The term “functional viral sequence” as used herein refers to any nucleic acid sequence (DNA or RNA) with functional activity such as enhancers, promoters, polyadenylation sites, sites of action of trans-acting factors, internal ribosome entry sites (IRES), translation frameshift sites, packaging sequences, integration sequences, or splicing sequences. If a drug were to target more than one functional viral sequence or viral gene product then patient-derived segments corresponding to each said viral gene would be inserted in the resistance test vector. In the case of combination therapy where two or more anti-virals targeting two different functional viral sequences or viral gene products are being evaluated, patient-derived segments corresponding to each functional viral sequence or viral gene product would be inserted in the resistance test vector. The patient-derived segments are inserted into unique restriction sites or specified locations, called patient sequence acceptor sites, in the indicator gene viral vector or for example, a packaging vector depending on the particular construction being used as described herein.

As used herein, “patient-derived segment” encompasses segments derived from human and various animal species. Such species include, but are not limited to chimpanzees and other primates, horses, cattles, cats and dogs.

Patient-derived segments can also be incorporated into resistance test vectors using any of several alternative cloning techniques. For example, cloning via the introduction of class II restriction sites into both the plasmid backbone and the patient-derived segments or by uracil DNA glycosylase primer cloning, or by site specific recombination, or by exonuclease overhang cloning.

The patient-derived segment may be obtained by any method of molecular cloning or gene amplification, or modifications thereof, by introducing patient sequence acceptor sites, as described below, at the ends of the patient-derived segment to be introduced into the resistance test vector. For example, in a gene amplification method such as PCR, restriction sites corresponding to the patient-sequence acceptor sites can be incorporated at the ends of the primers used in the PCR reaction. Similarly, in a molecular cloning method such as cDNA cloning, said restriction sites can be incorporated at the ends of the primers used for first or second strand cDNA synthesis, or in a method such as primer-repair of DNA, whether cloned or uncloned DNA, said restriction sites can be incorporated into the primers used for the repair reaction. The patient sequence acceptor sites and primers are designed to improve the representation of patient-derived segments. Sets of resistance test vectors having designed patient sequence acceptor sites provide representation of patient-derived segments that would be underrepresented in one resistance test vector alone.

Resistance test vectors systems are prepared by modifying an indicator gene viral vector (described below), or packaging vector, by introducing patient sequence acceptor sites, amplifying or cloning patient-derived segments and inserting the amplified or cloned sequences precisely into indicator gene viral vectors, or packaging vectors, at the patient sequence acceptor sites. Resistance test vector systems that are constructed from indicator gene viral vectors are in turn derived from genomic viral vectors or subgenomic viral vectors and an indicator gene cassette, each of which is described below. Resistance test vector systems that are constructed from packaging indicator vectors are in turn derived from genomic packaging vectors or subgenomic packaging vectors and an indicator gene cassette, each of which is described below. Resistance test vectors are then introduced into a host cell. Alternatively, a resistance test vector (also referred to as a resistance test vector system) is prepared by introducing patient sequence acceptor sites into a packaging vector, amplifying or cloning patient-derived segments and inserting the amplified or cloned sequences precisely into the packaging vector at the patient sequence acceptor sites and co-transfecting this packaging vector with an indicator gene viral vector.

In one preferred embodiment, the resistance test vector may be introduced into packaging host cells together with packaging expression vectors, as defined below, to produce resistance test vector viral particles that are used in drug resistance and susceptibility tests that are referred to herein as a “particle-based test.” In an alternative preferred embodiment, the resistance test vector may be introduced into a host cell in the absence of packaging expression vectors to carry out a drug resistance and susceptibility test that is referred to herein as a “non-particle-based test.”

As used herein a “packaging expression vector” provides the factors, such as packaging proteins (e.g. structural proteins such as core and envelope polypeptides), transacting factors, or genes required by replication-defective virus. In such a situation, a replication-competent viral genome is enfeebled in a manner such that it cannot replicate on its own. This means that, although the packaging expression vector can produce the trans-acting or missing genes required to rescue a defective viral genome present in a cell containing the enfeebled genome, the enfeebled genome cannot rescue itself.

Indicator or Indicator Gene

“Indicator or indicator gene” refers to a nucleic acid encoding a protein, DNA or RNA structure that either directly or through a reaction gives rise to a measurable or noticeable aspect, e.g. a color or light of a measurable wavelength or in the case of DNA or RNA used as an indicator a change or generation of a specific DNA or RNA structure. Preferred examples of an indicator gene is the E. coli lacZ gene which encodes beta-galactosidase, the luc gene which encodes luciferase either from, for example, Photonis pyralis (the firefly) or Renilla reniformis (the sea pansy), the E. coli phoA gene which encodes alkaline phosphatase, green fluorescent protein, the bacterial CAT gene which encodes chloramphenicol acetyltransferase, and the bacterial S-lactamase gene. Additional preferred examples of an indicator gene are secreted proteins or cell surface proteins that are readily measured by assay, such as radioimmunoassay (RIA), or fluorescent activated cell sorting (FACS), including, for example, growth factors, cytokines and cell surface antigens (e.g. growth hormone, Il-2 or CD4, respectively). “Indicator gene” is understood to also include a selection gene, also referred to as a selectable marker. Examples of suitable selectable markers for mammalian cells are dihydrofolate reductase (DHFR), thymidine kinase or E. coli gpt or genes that codes for resistance to the antibiotics hygromycin, neomycin, puromycin or zeocin. In the case of the foregoing examples of indicator genes, the indicator gene and the patient-derived segment are discrete, i.e. distinct and separate genes. In some cases a patient-derived segment may also be used as an indicator gene. In one such embodiment in which the patient-derived segment corresponds to more than one viral gene which is the target of an anti-viral, one of said viral genes may also serve as the indicator gene. For example, the HCV protease gene may serve as an indicator gene by virtue of its ability to cleave a chromogenic substrate or its ability to activate an inactive zymogen which in turn cleaves a chromogenic substrate, giving rise in each case to a color reaction. In a second example, the HCMV phosphotransferase gene may serve as an indicator gene by virtue of its ability to phosphorylate a substrate thereby up-regulating or down-regulating its activity. In all of the above examples of indicator genes, the indicator gene may be either “functional” or “non-functional” but in each case the expression of the indicator gene in the target cell is ultimately dependent upon the action of the patient-derived segment.

Functional Indicator Gene

In the case of a “functional indicator gene” the indicator gene may be capable of being expressed in a “packaging host cell/resistance test vector host cell” as defined below, independent of the patient-derived segment, however the functional indicator gene could not be expressed in the target host cell, as defined below, without the production of functional resistance test vector particles and their effective infection of the target host cell. In one embodiment of a functional indicator gene, the indicator gene cassette, comprising control elements and a gene encoding an indicator protein, is inserted into the indicator gene viral vector, or packaging viral vector, with the same or opposite transcriptional orientation as the native or foreign enhancer/promoter of the viral vector. One example of a functional indicator gene in the case of HCV, places the indicator gene and its promoter (a CMV IE enhancer/promoter) in the same or opposite transcriptional orientation as the HCV enhancer-promoter, respectively, or the T7 phage RNA polymerase promoter (herein referred to as T7 promoter) associated with the viral vector.

Non-functional Indicator Gene

Alternatively the indicator gene, may be “non-functional” in that the indicator gene is not efficiently expressed in a packaging host cell transfected with the resistance test vector, which is then referred to a resistance test vector host cell, until it is converted into a functional indicator gene through the action of one or more of the patient-derived segment products. An indicator gene is rendered non-functional through genetic manipulation according to this invention.

1. Permuted Promoter In one embodiment an indicator gene is rendered non-functional due to the location of the promoter, in that, although the promoter is in the same transcriptional orientation as the indicator gene, it follows rather than precedes the indicator gene coding sequence. This misplaced promoter is referred to as a “permuted promoter.” The non-functional indicator gene and its permuted promoter is rendered functional by the action of one or more of the viral proteins. One example of a non-functional indicator gene with a permuted promoter in the case of HCMV, places a promoter in the “b” region and the IRES, coding and terminating regions of the indicator gene in the c′/and/or adjacent U_{S} region. The non-functional indicator gene in the resistance test vector is converted into a functional indicator gene inversion of the U_{L}/U_{S} junction upon infection of the target cells, resulting from the repositioning of the CMV IE promoter relative to the indicator gene coding region. Following the inversion, properly arranged indicator genes are expressed in the target cell.

A permuted promoter may be any eukaryotic or prokaryotic promoter which can be transcribed in the target host cell. In one example, the CMV IE promoter/enhancer region can be used. In a second example the promoter will be small in size to enable insertion in the viral genome without disturbing viral replication. More preferably, a promoter that is small in size and is capable of transcription by a single subunit RNA polymerase introduced into the target host cell, such as a bacteriophage promoter, will be used. Examples of such bacteriophage promoters and their cognate RNA polymerases include those of phages T7, T3 and Sp6. A nuclear localization sequence (NLS) may be attached to the RNA polymerase to localize expression of the RNA polymerase to the nucleus where they may be needed to transcribed the repaired indicator gene. Such an NLS may be obtained from any nuclear-transported protein such as the SV40 T antigen. If a phage RNA polymerase is employed, an internal ribosome entry site (IRES) such as the EMC virus 5′ untranslated region (UTR) may be added in front of the indicator gene, for translation of the transcripts which are generally uncapped. In the case of HCMV, the permuted promoter can be introduced at any position that does not disrupt the cis acting elements that are necessary for HCMV DNA replication. Blocking sequences may be added at the ends of the resistance test vector should there be inappropriate expression of the non-functional indicator gene due to transfection artifacts (DNA concatenation). In the HCMV example of the permuted T7 promoter given above, such a blocking sequence may consist of a T7 transcriptional terminator, positioned to block readthrough transcription resulting from DNA concatenation.

2. Permuted Coding Region In a second embodiment, an indicator gene is rendered non-functional due to the relative location of the 51 and 3′ coding regions of the 5′ indicator gene, in that, the 3′ coding region precedes rather than follows the 5′ coding region. This misplaced coding region is referred to as a “permuted coding region.” The orientation of the non-functional indicator gene may be the same or opposite to that of the native or foreign promoter/enhancer of the viral vector, as mRNA coding for a functional indicator gene will be produced in the event of either orientation. The non-functional indicator gene and its permuted coding region is rendered functional by the action of one or more of the patient-derived segment products. An example of a non-functional indicator gene with a permuted coding region in the case of HCMV, places a 5′ indicator gene coding region with an associated promoter in the b region and a 3′ indicator gene coding region in the c′ region and/or adjacent U_{S} region of the HCMV genome, with the coding region having the opposite transcriptional orientation. In both examples, the 5′ and 3′ coding regions may also have associated splice donor and acceptor sequences, respectively, which may be heterologous or artificial splicing signals. The indicator gene cannot be functionally transcribed either by the associated promoter or viral promoters, as the permuted coding region prevents the formation of functional transcripts. The non-functional indicator gene in the resistance test vector is converted into a functional indicator gene by inversion of the U_{L}/U_{S} junction upon infection of the target cells, resulting from the repositioning of the 5′ and 3′ indicator gene coding regions relative to one another. Following transcription by the promoter associated with the 5′ coding region, RNA with appropriately arranged 5′ and 3′ coding regions produce a functional indicator gene product.

3. Negative strand RNA coding region. In a third embodiment, an indicator gene is rendered non-functional by virtue of the fact that it is expressed from RNA that is negative sense with respect to the virally encoded gene products. Expression of luciferase from mini-genome RNAs containing the luc gene in reverse orientation requires negative strand RNA made during virus replication (FIG. 5).

Indicator Gene Viral Vector—Construction

As used herein, “indicator gene viral vector” refers to a vector(s) comprising an indicator gene and its control elements and one or more viral genes. The indicator gene viral vector is assembled from an indicator gene cassette and a “viral vector,” defined below. The indicator gene viral vector may additionally include an enhancer, splicing signals, polyadenylation sequences, transcriptional terminators, or other regulatory sequences. Additionally the indicator gene viral vector may be functional or nonfunctional. In the event that the viral segments which are the target of the anti-viral drug (which for drug resistance and susceptibility testing are patient derived) are not included in the indicator gene viral vector they are provided in a second vector, which may be a packaging viral vector. An “indicator gene cassette” comprises an indicator gene and control elements. “Viral vector” refers to a vector comprising some or all of the following: viral genes encoding a gene product, control sequences, viral packaging sequences. The viral vector may additionally include one or more viral segments one or more of which may be the target of an anti-viral drug. Two examples of a viral vector which contain viral genes are referred to herein as an “genomic viral vector” and a “subgenomic viral vector.” A “genomic viral vector” is a vector which may comprise a deletion of a one or more viral genes to render the virus replication incompetent, but which otherwise preserves the mRNA expression and processing characteristics of the complete virus.

In one embodiment for an HCV drug susceptibility and resistance test, the genomic viral vector comprises C, E1, E2, NS2, NS3, NS4, and NS5 (See infra, pages 52-53). In one embodiment for an HCMV drug susceptibility and resistance test, the genomic viral vector comprises viruses deleted in one or a few genes such as JL54, UL80, UL97. A “subgenomic viral vector” refers to a vector comprising the coding region of one or more viral genes which may encode the proteins that are the target(s) of the anti-viral drug. In the case of HCV, a preferred embodiment is a subgenomic viral vector comprising the HCV NS2, NS3, NS4, NS5 genes (FIG. 6). In the case of HCMV, a preferred embodiment is a subgenomic viral vector comprising the HCMV amplicon plasmids containing one or a few viral genes such as UL54, UL80, UL90. Examples of viral clones used for viral vector construction are: Towne, Toledo, and AD169. The viral coding genes may be under the control of a native enhancer/promoter or a foreign viral or cellular enhancer/promoter. A preferred embodiment for an HCV drug susceptibility and resistance test, is to place the genomic or subgenomic viral coding regions under the control of the T7 promoter. A preferred embodiment for an HCMVV drug susceptibility and resistance test, is to place the genomic or subgenomic viral coding regions under the control of the endogenous HCMV promoters. In the case of an indicator gene viral vector that contains one or more viral genes which are the targets or encode proteins which are the targets of an anti-viral drug(s) then said vector contains the patient sequence acceptor sites. The patient-derived segments are inserted in the patient sequence acceptor site in the indicator gene viral vector which is then referred to as the resistance test vector, as described above.

“Patient sequence acceptor sites” are sites in a vector for insertion of patient-derived segments and said sites may be: 1) unique restriction sites introduced by site-directed mutagenesis into a vector; 2) naturally occurring unique restriction sites in the vector; or 3) selected sites into which a patient-derived segment may be inserted using alternative cloning methods (e.g. UDG cloning, exonuclease overhang cloning), 4) site specific recombination target sites. In one embodiment the patient sequence acceptor site is introduced into the indicator gene viral vector. The patient sequence acceptor sites are preferably located within or near the coding region of the viral protein which is the target of the anti-viral drug. The viral sequences used for the introduction of patient sequence acceptor sites are preferably chosen so that no change, or a conservative change, is made in the amino acid coding sequence found at that position. Preferably the patient sequence acceptor sites are located within a relatively conserved region of the viral genome to facilitate introduction of the patient-derived segments. Alternatively, the patient sequence acceptor sites are located between functionally important genes or regulatory sequences. Patient-sequence acceptor sites may be located at or near regions in the viral genome that are relatively conserved to permit priming by the primer used to introduce the corresponding restriction site into the patient-derived segment. To improve the representation of patient-derived segments further, such primers may be designed as degenerate pools to accommodate viral sequence heterogeneity, or may incorporate residues such as deoxyinosine (I) which have multiple base-pairing capabilities. Sets of resistance test vectors having patient sequence acceptor sites that define the same or overlapping restriction site intervals may be used together in the drug resistance and susceptibility tests to provide representation of patient-derived segments that contain internal restriction sites identical to a given patient sequence acceptor site, and would thus be underrepresented in either resistance test vector alone.

Host Cells

The resistance test vector is introduced into a host cell. Suitable host cells are mammalian cells. Preferred host cells are derived from human tissues and cells which are the principle targets of viral infection. In the case of HCV these include human cells such as hepatocytes, hepatoma cell lines and other cells. In the case of HCMV, suitable host cells include MRC5, HF, human foreskin fibroblasts and other cells. Human derived host cells will assure that the anti-viral drug will enter the cell efficiently and be converted by the cellular enzymatic machinery into the metabolically relevant form of the anti-viral inhibitor. Host cells are referred to herein as a “packaging host cells,” “resistance test vector host cells,” or “target host cells.” A “packaging host cell” refers to a host cell that provides the trans-acting factors and viral packaging proteins required by the replication defective viral vectors used herein, such as the resistance test vectors, to produce resistance test vector viral particles. The packaging proteins may be provided for by the expression of viral genes contained within the resistance test vector itself, a packaging expression vector(s), or both. A packaging host cell is a host cell which is transfected with one or more packaging expression vectors and when transfected with a resistance test vector is then referred to herein as a “resistance test vector host cell” and is sometimes referred to as a packaging host cell/resistance test vector host cell. Preferred host cells for use as packaging host cells for HCV include huh7, HepG2. Preferred host cells for use as packaging host cells for HCMV include MRC5 and HF. A “target host cell” refers to a cell to be infected by resistance test vector viral particles produced by the resistance test vector host cell in which expression or inhibition of the indicator gene takes place. Preferred host cells for use as target host cells for HCV include HepG2 (Hiramatsu et al. (1997), J.Viral Hepatol. 4(suppl.1), 61-67), Huh7 (Yoo et al. (1995), J. Virol. 69, 32-38), Vero (Valli et al. (1997) Res. Virol. 148, 181-186), Molt4Ma (Shimizu et al. (1992), PNAS 89, 5477-5481), HPBMa (Shimizu et al. (1993), PNAS 90, 6037-6041; Shimizu and Yoshikura (1994), J. Virol. 68, 8406-8408; Shimizu et al. (1994), J. Virol. 68, 1494-1500), MT-2 (Mizutani et al. (1996), J. Virol. 70, 7219-7223). Preferred host cells for use as target host cells for HCMV include MRC5 and HF.

Drug Susceptibility and Resistance Tests

The drug susceptibility and resistance tests of this invention may be carried out in one or more host cells. Viral drug susceptibility is determined as the concentration of the anti-viral agent at which a given percentage of indicator gene expression is inhibited (e.g. the IC50 for an anti-viral agent is the concentration at which 50% of indicator gene expression is inhibited). A standard curve for drug susceptibility of a given anti-viral drug can be developed for a viral segment that is either a standard laboratory viral segment or from a drug-naive patient (i.e. a patient who has not received any anti-viral drug) using the method of this invention. Correspondingly, viral drug resistance is a decrease in viral drug susceptibility for a given patient either by comparing the drug susceptibility to such a given standard or by making sequential measurement in the same patient over time, as determined by increased inhibition of indicator gene expression (i.e. decreased indicator gene expression).

In the first type of drug susceptibility and resistance test, resistance test vector viral particles are produced by a first host cell (the resistance test vector host cell) that is prepared by transfecting a packaging host cell with the resistance test vector and packaging expression vector(s). The resistance test vector viral particles are then used to infect a second host cell (the target host cell) in which the expression of the indicator gene is measured. Such a two cell system comprising a packaging host cell which is transfected with a resistance test vector, which is then referred to as a resistance test vector host cell, and a target cell are used in the case of either a functional or non-functional indicator gene. Functional indicator genes are efficiently expressed upon transfection of the packaging host cell and would require infection of a target host cell with resistance test vector host cell supernatant to carry out the test of this invention. Non-functional indicator genes with a permuted promoter, a permuted coding region, or an negative sense strand indicator RNA, are not efficiently expressed upon transfection of the packaging host cell and thus the infection of the target host cell can be achieved either by co-cultivation by the resistance test vector host cell and the target host cell or through infection of the target host cell using the resistance test vector host cell supernatant. In the second type of drug susceptibility and resistance test, a single host cell (the resistance test vector host cell) also serves as a target host cell. The packaging host cells are transfected and produce resistance test vector viral particles and some of the packaging host cells also become the target of infection by the resistance test vector particles. Drug susceptibility and resistance tests employing a single host cell type are possible with viral resistance test vectors comprising a non-functional indicator gene with a permuted promoter, a permuted coding region, or negative sense strand indicator RNA. Such indicator genes are not efficiently expressed upon transfection of a first cell, but are only efficiently expressed upon infection of a second cell, and thus provide an opportunity to measure the effect of the anti-viral agent under evaluation. In the case of a drug susceptibility and resistance test using a resistance test vector comprising a functional indicator gene, neither the co-cultivation procedure nor the resistance and susceptibility test using a single cell type can be used for the infection of target cells (is this true for HCMV). A resistance test vector comprising a functional indicator gene requires a two cell system using filtered supernatants from the resistance test vector host cells to infect the target host cell.

In one embodiment of the invention in the case of HCV, a particle-based resistance tests are carried out with resistance test vectors derived from genomic viral vectors, i.e., pXHCV-luc; pXHCV-IRESluc; pXHCV/pxIRESluc; pXHCV/pXASIRESluc; pXluc-NSHCV/pXsHCV; pXBVDV(HCVNS3)luc; pXBVDV(HCVNS5B)luc. In one embodiment of the invention in the case of HCMV, a particle-based resistance tests are carried out with resistance test vectors derived from genomic viral vectors, i.e., pA-CMV-VS-geneX; pA-CMV-CS-genex; pA-CMV-VS-geneX-(NF-IG)PP/pA-CMV-CS-geneX-(NF-IG)PP; pA-CMV-VS-geneX-(NF-IG) PCR/pA-CMV-CS-geneX-(NF-IG) PCR; pA-CMV-VS-geneX-F-IG/pA-CMV-CS-geneX-F-IG, which are cotransfected with the packaging expression vector HCMVβ_{2.7}FIG/ΔgeneX or HCMVΔgeneX.

In the case of the particle-based susceptibility and resistance test, resistance test vector viral particles are produced by a first host cell (the resistance test vector host cell) that is prepared by transfecting a packaging host cell with the resistance test vector and packaging expression vector(s). The resistance test vector viral particles are then used to infect a second host cell (the target host cell) in which the expression of the indicator gene is measured. In a second type of particle-based susceptibility and resistance test, a single host cell type (the resistance test vector host cell) serves both purposes: some of the packaging host cells in a given culture are transfected and produce resistance test vector viral particles and some of the host cells in the same culture are the target of infection by the resistance test vector particles thus produced. Resistance tests employing a single host cell type are possible with resistance test vectors comprising a non-functional indicator gene with a permuted promoter since such indicator genes are efficiently expressed upon infection of a permissive host cell, they are not efficiently expressed upon transfection of the same host cell type, and thus provide an opportunity to measure the effect of the anti-viral agent under evaluation. For similar reasons, resistance tests employing two cell types may be carried out by co-cultivating the two cell types as an alternative to infecting the second cell type with viral particles obtained from the supernatants of the first cell type.

The packaging host cells are transfected with the resistance test vector and the appropriate packaging expression vector(s) to produce resistance test vector host cells. Individual anti-viral agents for HCV, including the protease inhibitors, IRES inhibitors, and the polymerase inhibitors as well as combinations thereof, are added to individual plates of packaging host cells at the time of their transfection, at an appropriate range of concentrations. Twenty-four to 48 hours after transfection, target host cells are infected by co-cultivation with resistance test vector host cells or with resistance test vector viral particles obtained from filtered supernatants of resistance test vector host cells. Each anti-viral agent, or combination thereof, is added to the target host cells prior to or at the time of infection to achieve the same final concentration of the given agent, or agents, present during the transfection.

Determination of the expression or inhibition of the indicator gene in the target host cells infected by co-cultivation or with filtered viral supernatants is made by assay of indicator gene expression, for example in the case where the indicator gene is the firefly luc gene, by measuring luciferase activity. The reduction in luciferase activity observed for target host cells infected with a given preparation of resistance test vector viral particles in the presence of a given antiviral agent, or agents, as compared to a control run in the absence of the antiviral agent, generally relates to the log of the concentration of the antiviral agent as a sigmoidal curve. This inhibition curve is used to calculate the apparent inhibitory concentration (IC) of that agent, or combination of agents, for the viral target product encoded by the patient-derived segments present in the resistance test vector.

In the case of a one cell susceptibility and resistance test, host cells are transfected with the resistance test vector and the appropriate packaging expression vector(s) to produce resistance test vector host cells. Individual antiviral agents, or combinations thereof, are added to individual plates of transfected cells at the time of their transfection, at an appropriate range of concentrations. At an appropriate time after transfection, cells are collected and assayed for firefly luciferase activity. As transfected cells in the culture do not efficiently express the indicator gene, transfected cells in the culture, as well superinfected cells in the culture, can serve as target host cells for indicator gene expression. The reduction in luciferase activity observed for cells transfected in the presence of a given antiviral agent, or agents as compared to a control run in the absence of the antiviral agent(s), generally relates to the log of the concentration of the antiviral agent as a sigmoidal curve. This inhibition curve is used to calculate the apparent inhibitory concentration (IC) of an agent, or combination of agents, for the viral target product encoded by the patient-derived segments present in the resistance test vector.

Antiviral Drugs/Drug Candidates

The antiviral drugs being added to the test system are added at selected times depending upon the target of the antiviral drug. For example, in the case of HCV protease inhibitors, they are added to individual plates of packaging host cells at the time of their transfection with a resistance test vector, at an appropriate range of concentrations. HCV protease inhibitors may also be added to the target host cells at the time of infection to achieve the same final concentration added during transfections. For HCMV, phosphotransferase, DNA polymerase and protease inhibitors, including GCV, cidofovir, foscarnet are added to individual plates of target host cells at the time of transfection/infection by the resistance test vector viral particles, at a test concentration. Alternatively, the antiviral drugs may be present throughout the assay. The test concentration is selected from a range of concentrations which is designed to give a satisfactory inhibition profile for resistant and sensitive isolates.

In another embodiment of this invention, a candidate antiviral compound is tested in the drug susceptibility and resistance test of this invention. The candidate antiviral compound is added to the test system at an appropriate concentration and at selected times depending upon the protein target of the candidate anti-viral. Alternatively, more than one candidate antiviral compound may be tested or a candidate antiviral compound may be tested in combination with an approved antiviral drug such as GCV for HCMV or a compound which is undergoing clinical trials. The effectiveness of the candidate antiviral will be evaluated by measuring the expression or inhibition of the indicator gene. In another aspect of this embodiment, the drug susceptibility and resistance test may be used to screen for viral mutants. Following the identification of resistant mutants to either known anti-virals or candidate anti-virals the resistant mutants are isolated and the DNA is analyzed. A library of viral resistant mutants can thus be assembled enabling the screening of candidate anti-virals, alone or in combination. This will enable one of ordinary skill to identify effective anti-virals and design effective therapeutic regimens.

General Materials and Methods

Most of the techniques used to construct vectors, and transfect and infect cells, are widely practiced in the art, and most practitioners are familiar with the standard resource materials which describe specific conditions and procedures. However, for convenience, the following paragraphs may serve as a guideline.

“Plasmids” and “vectors” are designated by a lower case p followed by letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

Construction of the vectors of the invention employs standard ligation and restriction techniques which are well understood in the art (see Ausubel et al., (1987) Current Protocols in Molecular Biology, Wiley—Interscience or Maniatis et al., (1992) in Molecular Cloning: A laboratory Manual, Cold Spring Harbor Laboratory, N.Y.). Isolated plasmids, DNA sequences, or synthesized oligonucleotides are cleaved, tailored, and relegated in the form desired. The sequences of all DNA constructs incorporating synthetic DNA were confirmed by DNA sequence analysis (Sanger et al. (1977) Proc. Natl. Acad. Sci. 74, 5463-5467).

“Digestion” of DNA refers to catalytic cleavage of the DNA with a restriction enzyme that acts only at certain sequences, restriction sites, in the DNA. The various restriction enzymes used herein are commercially available and their reaction conditions, cofactors and other requirements are known to the ordinarily skilled artisan. For analytical purposes, typically 1 μg of plasmid or DNA fragment is used with about 2 units of enzyme in about 20 μl of buffer solution. Alternatively, an excess of restriction enzyme is used to insure complete digestion of the DNA substrate. Incubation times of about one hour to two hours at about 37° C. are workable, although variations can be tolerated. After each incubation, protein is removed by extraction with phenol/chloroform, and may be followed by ether extraction, and the nucleic acid recovered from aqueous fractions by precipitation with ethanol. If desired, size separation of the cleaved fragments may be performed by polyacrylamide gel or agarose gel electrophoresis using standard techniques. A general description of size separations is found in Methods of Enzymology 65:499-560 (1980).

Restriction cleaved fragments may be blunt ended by treating with the large fragment of E. coli DNA polymerase I (Klenow) in the presence of the four deoxynucleotide triphosphates (dNTPs) using incubation times of about 15 to 25 minutes at 200 C. in 50 mM Tris (pH 7.6) 50 mM NaCl, 6 mM MgCl2, 6 mM DTT and 5-10 micromole dNTPs. The Klenow fragment fills in at 5′ sticky ends but chews back protruding 3′ single strands, even though the four dNTPs are present. If desired, selective repair can be performed by supplying only one of the dNTPs, or with selected dNTPs, within the limitations dictated by the nature of the sticky ends. After treatment with Klenow, the mixture is extracted with phenol/chloroform and ethanol precipitated. Treatment under appropriate conditions with Si nuclease or Bal-31 results in hydrolysis of any single-stranded portion.

Ligations are performed in 15-50 μl volumes under the following standard conditions and temperatures: 20 mM Tris-Cl pH 7.5, 10 mM MgCl2, 10 mM DTT, 33 mg/ml BSA, 10 mM-50 mM NaCl, and either 40 AM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 33-100 μg/ml total DNA concentrations (5-100 mM total end concentration). Intermolecular blunt end ligations (usually employing a 10-30 fold molar excess of linkers) are performed at 1 μM total ends concentration.

“Transient expression” refers to unamplified expression within about one day to two weeks of transfection. The optimal time for transient expression of a particular desired heterologous protein may vary depending on several factors including, for example, any transacting factors which may be employed, translational control mechanisms and the host cell. Transient expression occurs when the particular plasmid that has been transfected functions, i.e., is transcribed and translated. During this time the plasmid DNA which has entered the cell is transferred to the nucleus. The DNA is in a nonintegrated state, free within the nucleus. Transcription of the plasmid taken up by the cell occurs during this period. Following transfection the plasmid DNA may become degraded or diluted by cell division. Random integration within the cell chromatin occurs.

In general, vectors containing promoters and control sequences which are derived from species compatible with the host cell are used with the particular host cell. Promoters suitable for use with prokaryotic hosts illustratively include the beta-lactamase and lactose promoter systems, alkaline phosphatase, the tryptophan (trp) promoter system and hybrid promoters such as tac promoter. However, other functional bacterial promoters are suitable. In addition to prokaryotes, eukaryotic microbes such as yeast cultures may also be used. Saccharomyces cerevisiae, or common baker's yeast is the most commonly used eukaryotic microorganism, although a number of other strains are commonly available. Promoters controlling transcription from vectors in mammalian host cells may be obtained from various sources, for example, the genomes of viruses such as: polyoma, simian virus 40 (SV40), adenovirus, retroviruses, hepatitis B virus and preferably cytomegalovirus, or from heterologous mammalian promoters, e.g. β-actin promoter. The early and late promoters of the SV 40 virus are conveniently obtained as an SV40 restriction fragment which also contains the SV40 viral origin of replication. The immediate early promoter of the human cytomegalovirus is conveniently obtained as a HindIII E restriction fragment. Of course, promoters from the host cell or related species also are useful herein.

The vectors used herein may contain a selection gene, also termed a selectable marker. A selection gene encodes a protein, necessary for the survival or growth of a host cell transformed with the vector. Examples of suitable selectable markers for mammalian cells include the dihydrofolate reductase gene (DHFR), the ornithine decarboxylase gene, the multi-drug resistance gene (mdr), the adenosine deaminase gene, and the glutamine synthase gene. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. The second category is referred to as dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern and Berg (1982) J. Molec. Appl. Genet. 1, 327), mycophenolic acid (Mulligan and Berg (1980) Science 209, 1422), or hygromycin (Sugden et al. (1985) Mol. Cell. Biol. 5, 410-413). The three examples given above employ bacterial genes under eukaryotic control to convey resistance to the appropriate drug neomycin (G418 or genticin), xgpt (mycophenolic acid) or hygromycin, respectively.

“Transfection” means introducing DNA into a host cell so that the DNA is expressed, whether functionally expressed or otherwise; the DNA may also replicate either as an extrachromosomal element or by chromosomal integration. Unless otherwise provided, the method used herein for transformation of the host cells is the calcium phosphate co-precipitation method of Graham and van der Eb (1973) Virology 52, 456-457. Alternative methods for transfection are electroporation, the DEAE-dextran method, lipofection and biolistics (Kriegler (1990) Gene Transfer and Expression: A Laboratory Manual, Stockton Press).

Host cells may be transfected with the expression vectors of the present invention and cultured in conventional nutrient media modified as is appropriate for inducing promoters, selecting transformants or amplifying genes. Host cells are cultured in F12:DMEM (Gibco) 50:50 with added glutamine and without antibiotics. The culture conditions, such as temperature, pH and the like, are those previously used with the host cell selected for expression, and will be apparent to the ordinarily skilled artisan.

The following examples merely illustrate the best mode now known for practicing the invention, but should not be construed to limit the invention. All literature references throughout this application are expressly incorporated by reference.

### EXAMPLE 1

HCV Drug Susceptibility and Resistance Test Using Resistance Test Vectors Comprising Patient-derived Segment(s) and a Functional Indicator Gene Fused to the HCV Polyprotein.

Indicator Gene Viral Vector—Construction

The indicator gene viral vector (IGVV) pXHCV-luc was designed using HCV genomic viral vectors containing a functional indicator gene fused to the HCV polyprotein. The IGVV is constructed by inserting the open reading frame for the indicator gene in a cDNA construct containing the entire HCV genome producing an in-frame fusion protein. The IGVV also contains all the cis-acting regulatory elements in the 5′ and 3′ untranslated regions (UTRs) required for replication, transcription, and translation of the HCV RNA. In one embodiment, the luciferase open reading frame is placed immediately downstream of the NS5 coding region, with a spacer region containing the recognition sequence for the NS3/4A protease (FIG. 3A). An example of such a cleavage site is TEDVVCC-SMSYTWT, representing the junction between NS5A and NS5B (Grakoui et al 1993, J. Virol. 67:2832; Steinkuhler et al., 1996, J. Virol. 70:6694). The expected cleavage products are HCV NS5B containing a C-terminal extension (e.g., TEDVVCC), and luciferase containing an N-terminal extension (e.g., SMSYTWT). In a second embodiment, the luciferase open reading frame is placed between the NS5A and NS5B open reading frames, with an NS5A-5B cleavage sequence at both the N-terminal NS5A-luc and C-terminal luc-NS5B junctions. The luciferase protein produced from this construct contains an N-terminal SMSYTWT and a C-terminal TEDVVCC extension. In a third embodiment, the luciferase open reading frame is placed between the NS4B and NS5A open reading frames, with an NS4B-5A cleavage sequence (SECTTPC-SGSWLRD) at both the N-terminal NS4B-luc and C-terminal luc-NS5A junctions. The luciferase protein produced from this construct contains an N-terminal SGSWLRD and a C-terminal SECTTPC extension. In a fourth embodiment, the luciferase open reading frame is placed between the NS4A and NS4B open reading frames, with an NS4A-4B cleavage sequence (FDEMEEC-SQHLPYI) at both the N-terminal NS4A-luc and C-terminal luc-NS4B junctions. The luciferase protein produced from this construct contains an N-terminal SQHLPYI and a C-terminal FDEMEEC extension. The short extensions at the N and C-termini of luciferase or at the C-terminus of NS5B do not dramatically affect activity.

The viral vector is assembled from a full length cDNA construct of HCV, which consists of (in the 5′ to 3′ orientation) the 5′ UTR, the open reading frame for the 3010 amino acid polyprotein, and the 3′ UTR. The polyprotein contains within it the capsid (C) open reading frame, the envelope glycoprotein genes (E1 and E2), the NS2 (a cis-acting auto-protease that cleaves the polyprotein at a specific site at the NS2-NS3 junction), NS3 (helicase and serine protease), NS4A (required as a cofactor for NS3 activity), NS4B, NS5A, and NS5B (the RNA-dependent RNA polymerase) open reading frames. The luciferase open reading frame is also contained within the polyprotein open reading frame, located variously as described above.

In one embodiment, the IGVV contains a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme (e.g. see Chowrira et al. 1994, J. Biol. Chem. 269: 25864). The IGVV is transfected as DNA into cells expressing the RNA polymerase in the cytoplasm. Such expression is achieved by several methods including cotransfection with a polymerase expression vector, infection with a recombinant vaccinia virus expressing the polymerase (Fuerst et al. 1986, PNAS 83:8122), or by previously establishing a cell line permanently expressing the polymerase (see FIG. 3C). The IGVV additionally contains a poly-A or poly-U sequence immediately following the HCV 3′ terminus, so that the transcribed RNA contains a poly-A or poly-U tail at the 3′ end. In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator may be a specific sequence recognized by the bacteriophage RNA polymerase as a termination site or a self-cleaving ribozyme (see chowrira et al. (1994) J. Biol. Chem. 269, 25856-25864). Alternatively, the terminator is a restriction endonuclease site allowing for linearization of the DNA template prior to transcription (see FIG. 3D). In this case the vector also contains a poly-A or poly-U sequence at the 3′ end.

In transfected cells, the RNA is translated, using an internal initiation mechanism via the internal ribosome entry sequence (IRES), to yield the HCV polyprotein-luc fusion protein. Release of active luciferase from the HCV polyprotein fusion is dependent on the action of NS3/4A, itself expressed from the genomic RNA. High level expression takes place when the genomic RNA is replicated and amplified in the transfected cells, which is dependent on the action of the viral polymerase NS5B as well as the viral proteases NS2 and NS3/4A. In the case where the luciferase is inactive when it is part of the large HCV polyprotein, activity can be measured directly in the transfected cells since release of active luciferase is dependent on HCV RNA replication (one cell assay). In the case where luciferase has significant activity as a fusion protein, progeny virions will be collected and used to infect new target cells (two cell assay). Transfer of the IGVV RNA from the transfected cells to the infected target cells is dependent on replication and encapsidation of the RNA in the transfected cells, which in turn is dependent on the correct expression, processing and activity of the HCV viral structural and non-structural proteins. To augment the efficiency of transfer (i.e. packaging of the IGVV RNA into new virions) the target cells may be simultaneously infected with wild-type HCV virus or transfected with wild type HCV RNA or cDNA expression constructs. To further augment the replication and packaging of the IGVV RNA, input RNAs (see FIG. 3D) are cotransfected with purified NS5B protein (i.e. as RNP complexes), so that transcription can commence immediately upon uptake into the cells. This strategy or variation of it has been applied to the negative-stranded RNA viruses such as influenza virus (Enami and Palese 1991, J. Virol. 65: 2711-2713), rabies virus (Schnell et al. 1994, EMBO J. 13: 4195-4203), and vesicular stomatitis virus (Lawson et al. 1995, PNAS 92: 4477-4481).

Resistance Test Vectors—Construction

A resistance test vector (RTV) is constructed from the indicator gene viral vector by replacing a region of the HCV genome corresponding to the protein which is the anti-viral drug target (e.g. NS3/4A, NS5B, or the IRES) with the corresponding region derived from viruses and/or RNA present in the blood and/or cells of an infected patient (patient-derived segment, or PDS). In one embodiment, in the case of an NS3/4A protease inhibitor, the IGVV is modified by introducing unique restriction sites, called patient sequence acceptor sites (PSAS), in or near the NS3/4A genes (nucleotides 3418-5473 of the H strain of HCV). The patient derived segment obtained from the patient derived virus is then transferred into the PSAS in the IGVV (FIG. 3A). The wild-type NS3/4A region is removed from the IGVV by digestion with restriction endonucleases recognizing the patient sequence acceptor sites; these sequences are then replaced with DNA fragments generated by RT/PCR from patient-derived viral RNA obtained from plasma or serum or cells. The PCR products are generated using primers which contain the restriction endonuclease sites required for generation of compatible cohesive ends for cloning into the digested IGVV. RT and PCR primer binding sites are selected, and primer sequences designed, to enable amplification of as many different subtypes of HCV as possible. In a second embodiment, in the case of an inhibitor of the NS5B RDRP, the sequences spanning the NS5B open reading frame (nucleotides 7601-9373 of the H strain of HCV) are removed from the IGVV at unique patient sequence acceptor sites; these sequences are then replaced with the corresponding PDS generated by RT/PCR from patient viral RNA obtained from plasma or serum or cells. In a third embodiment, in the case of IRES inhibitors, the sequences spanning the IRES (nucleotides 1-709 of the H strain of HCV) are removed from the IGVV at unique patient sequence acceptor sites; these sequences are then replaced with corresponding PDS generated by RT/PCR from patient viral RNA obtained from plasma or serum or cells. The foregoing methods are applicable to other targets of anti-HCV drugs by identifying the gene encoding the target of the drug in the IGVV; introducing unique patient sequence acceptor sites into the IGVV; and replacing the target gene with a PDS.

Drug Susceptibility and Resistance Tests

Drug resistance and susceptibility tests are carried out with a resistance test vector prepared as described above (either as DNA or RNA) by transfection, using either a one cell assay or a two cell assay. Transfection of host cells with a resistance test vector produces HCV viral particles containing an encapsidated indicator gene RNA.

Replicate transfections are performed on a series of packaging host cell cultures maintained either in the absence of the anti-viral drug or in increasing concentrations of the anti-HCV drug (e.g., an HCV NS3/4A protease inhibitor, NS5B polymerase inhibitor, or IRES inhibitor). After maintaining the packaging host cells for several days in the presence or absence of the anti-HCV drug the level of drug susceptibility or resistance can be assessed by measuring indicator gene expression either directly in the host packaging cell lysates or in isolated HCV particles obtained by harvesting the host packaging cell culture media. Alternative approaches can be used to evaluate drug susceptibility and resistance in the cell lysates and the isolated HCV particles.

In one embodiment, referred to as the one cell assay, drug susceptibility or resistance is assessed by measuring luciferase expression or activity in the transfected packaging host cells in the presence or absence of anti-viral drug. A reduction in luciferase activity observed for cells transfected in the presence of a given anti-viral agent, or combination of agents as compared to a control run in the absence of the anti-viral agent(s), is used to calculate the inhibiting constant (Kᵢ) of that agent or to generate a sigmoid curve relating the log of the concentration of the anti-viral agent to luciferase activity.

In a second embodiment, referred to as the two cell assay, drug susceptibility or resistance is assessed by measuring luciferase gene expression and/or activity, in the target host cells following infection with HCV particles obtained by transfecting host cells. At the time of transfection or infection, depending on the drug target, the appropriate concentration of the anti-viral drug is added to the host or target cell cultures. Several days following the infection, the target host cells are lysed and luciferase expression is measured. A reduction in luciferase expression will be observed for cells infected in the presence of drugs which inhibit HCV replication, for example by inhibiting either the protease (NS3/4A) or RDRP (NS5B) activities of HCV as compared to a control run in the absence of drug.

In a third embodiment, in which changes in RNA structure are used as an indicator, drug susceptibility or resistance is assessed by measuring the level of HCV RNA replication that has occurred within the transfected host cells. In host cells transfected with the DNA of the HCV viral vector, RNA is transcribed (or alternatively, the RNA is transcribed in vitro and transfected directly) as positive (mRNA) sense RNA, which can then serve as a template for the production of negative sense cRNA by the action of NS5B polymerase. Alternatively, positive sense RNA is transfected, which is translated and serves as a template for negative sense cRNA synthesis. To measure HCV RNA replication, RNA is isolated from the transfected cells and treated with DNAse to remove residual input DNA (the DNAse treatment would not be absolutely required if the cells were transfected with positive sense RNA). An RT primer is designed to hybridize specifically to negative sense HCV RNA to prime the synthesis of positive sense cDNA; after RNAse digestion to prevent reverse transcription of positive sense RNA using the PCR primers, the cDNA is amplified by PCR.

Formation of the amplification target cDNA of positive sense within the transfected cells follows initiation of HCV RNA replication resulting in the formation of negative sense RNA. Anti-viral drugs that inhibit HCV RNA replication (RNA-dependent RNA polymerase activity), or production of an active form of the polymerase (by the NS3/4A protease), will limit the formation of the RNA target sequence, which is measured as a decrease in the amplified DNA product using any one of a number of quantitative amplification assays. In an alternative embodiment in which changes in RNA structure are used as the indicator, the 5′ exonuclease activity of the amplification enzyme (e.g. Taq polymerase) is measured rather than the production of amplified DNA (Heid et al., 1996, Genome Research 6:986-994). The 5′ exonuclease activity is measured by monitoring the nucleolytic cleavage of a fluorescently tagged oligonucleotide probe capable of binding to the amplified DNA template region flanked by the PCR primer binding sites. The performance of this assay is dependent on the close proximity of the 3′ end of the upstream primer to the 5′ end of the oligonucleotide probe. When the primer is extended it displaces the 5′ end of the oligonucleotide probe such that the 5′ exonuclease activity of the polymerase cleaves the oligonucleotide probe.

Drug Screening

Drug screening is carried out using an indicator gene viral vector containing a functional indicator gene fused to the HCV polyprotein. In transfected host cells, the indicator gene viral vector produces an RNA transcript containing the indicator gene (or alternatively, the RNA is transcribed in vitro and transfected directly). Translation of this RNA, or of mRNA produced as a result of replication and transcription by the viral RDRP (NS5B), produces the structural and enzymatic viral functions that are necessary for viral RNA replication and particle formation. The transfected cells give rise to HCV viral particles containing an encapsidated indicator gene viral vector RNA, which also contains the functional indicator gene fused to the HCV polyprotein gene.

Drug screening is performed as follows: indicator gene viral vector DNA or RNA is used to transfect host cells. Replicate transfections are performed on a series of packaging host cell cultures maintained either in the absence or presence of potential anti-viral compounds (e.g., candidate HCV NS3/4A protease or NS5B polymerase inhibitors). After maintaining the transfected host cells for up to several days in the presence or absence of the candidate anti-viral drugs the level of inhibition of RNA replication is assessed by measuring indicator gene expression either directly in the transfected host cell lysates, or in isolated HCV particles obtained by harvesting the host transfected cell culture media, or in target cells which are infected with the isolated HCV particles. Either RNA detection or indicator gene activity methods, described above, can be used to evaluate potential anti-HCV drug candidates.

### EXAMPLE 2

HCV Drug Susceptibility and Resistance Test Using Resistance Test Vectors Comprising Patient-derived Segment(s) and a Functional Indicator Gene Expressed From an Internal Ribosomal Initiation Sequence.

Indicator Gene Viral Vector—Construction

Initiation of translation of the HCV polyprotein occurs via a cap-independent internal initiation mechanism. The 5′ end of the viral RNA, comprising the untranslated region (UTR) and the first 369 nucleotides of the C open reading frame, contains a sequence and/or structure which directs cap-independent translation initiation (Tsukiyama-Kohara et al, J Virol. 66:1476, 1992; Wang et al, J Virol. 67:3338, 1993; Lu and Wimmer, PNAS 93:1412, 1996). Other viruses such as poliovirus (PV) (Pelletier and Sonenberg (1988), Nature, 334, 320-325), encephalomyocarditis virus (EMCV) (Jang et al. (1989), J. Virol. 63, 1651-1660), rhinovirus (RV) (Rohll et al. (1994), J. Virol. 68, 4384-4391), hepatitis A virus (HAV) (Brown et al. (1994), J. Virol. 68, 1066-1074; Glass et al. (1993) Virol. 193, 842-852), as well as the pestivirus, bovine viral diarrhea virus (BVDV) (Poole et al. (1995) Virology, 189, 285-292) to which HCV is closely related, employ similar mechanisms for translation initiation, altough the sequences which serve as the internal ribosome entry site (IRES) are different for each virus. Some cellular mRNAs are also known to initiate translation internally via an IRES (Macejak and Sarnow (1991), Nature, 353, 90-94). These RNA elements have been shown to be capable of directing translation initiation when located in between two open reading frames, as well as at the 5′ end of RNAs. These bicistronic RNAs can be used to obtain expression of two proteins from the same RNA by independently directing the translation of both open reading frames.

Indicator gene viral vectors containing a functional IG expressed from an internal ribosomal initiation sequence are constructed by inserting the open reading frame for an indicator gene, for example, luciferase, in a cDNA construct, containing the entire HCV genome, as a second cistronic element preceded by an IRES. Insertion of the IRES (either the native HCV 5′ UTR or that of another virus) and luciferase downstream of the HCV polyprotein provides for luciferase gene expression independently of that of HCV proteins (see FIG. 4). Note that when testing for resistance to a drug that inhibits the function of the HCV IRES, the IRES used for expression of luciferase must be derived from a virus other than HCV, which is not affected by the drug. The IGVV thus contains the following elements in a 5′ to 3′ orientation: a promoter sequence, the HCV 5′ UTR, the complete HCV polyprotein coding sequence, an IRES, hthe luciferase coding region, the HCV 3′ UTR, and a transcription terminator.

In one embodiment, the IGW contains a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme. The IGW is transfected as DNA into cells expressing the RNA polymerase in the cytoplasm. Such expression is achieved by several methods including cotransfection with a polymerase expression vector, infection with a recombinant vaccinia virus expressing the polymerase, or by previously establishing a cell line permanently expressing the polymerase (see FIG. 3C). The IGVV additionally contains a poly-A or poly-U sequence immediately following the HCV 3′ terminus, so that the transcribed RNA contains a poly-A or poly-U tail at the 3′ end. In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator may be a specific sequence recognized by the bacteriophage RNA polymerase as a termination site or a self-cleaving ribozyme. Alternatively, the terminator is a restriction endonuclease site allowing for linearization of the DNA template prior to transcription (see FIG. 3D). In this case the vector also contains a poly-A or poly-U sequence at the 3′ end.

Resistance Test Vectors—Construction

Resistance test vectors containing a functional indicator gene expressed from an internal ribosomal initiation sequence are constructed from IGVVs described above and patient-derived HCV sequences as described in Example 1. The IGVV is modified to include PSAS for the insertion of NS3/4A, NS5B, or IRES containing PDS (described in Example 1, see FIG. 3A).

Drug Susceptibility and Resistance Tests

Drug resistance and susceptibility tests are carried out with a resistance test vector prepared as described above (either as DNA or RNA) by transfection, using either a one cell or two cell assay. Transfection of host cells with a resistance test vector produces HCV viral particles containing an encapsidated indicator gene RNA. Drug resistance and susceptibility tests are performed as described in Example 1.

Drug Screening

Drug screening using an IGVV containing a functional indicator gene expressed from an internal ribosomal initiation sequence is performed essentially as described in Example 1 above.

### EXAMPLE 3

HCV Drug Susceptibility and Resistance Test Using Resistance Test Vectors Comprising Patient-derived Segment(s) and a Functional Indicator Gene Expressed From a Replication Defective Minigenome.

Indicator Gene Viral Vector—Construction

HCV replication-dependent expression of an indicator gene is achieved by constructing an artificial HCV subgenomic viral vector, or “minigenome”, consisting of the HCV 5′ UTR and, if required, an amino-terminal portion of the C open reading frame (required as part of the IRES), an IG, for example luciferase, and the HCV 3′ UTR (see FIG. 5). Luciferase is produced bearing an N-terminal extension derived from the C open reading frame; alternatively, the ATG at the beginning of the C open reading frame is mutated so that translation begins at the ATG of luciferase. The 5′ UTR plus the N-terminus of C and 3′ UTR contain all cis-acting signals required for translation, replication, and packaging of the RNA. The luciferase minigenome is co-transfected, either as DNA or RNA (see Example 1, FIGS. 3B-3D), with a full-length helper HCV genomic construct; replication and packaging into progeny viruses of the minigenome RNA is dependent on the HCV replication machinery, including the NS3/4A protease and NS5B RDRP, produced from the helper HCV genomic RNA, as well as of the cis-acting regulatory elements of the minigenome.

Indicator gene viral vectors comprising a functional indicator gene expressed from a replication defective minigenome and a helper HCV genomic construct are constructed as follows. The minigenome contains the following elements in a 5′ to 3′ orientation: a promoter sequence, the HCV 5′ UTR, the first 24 or 369 nucleotides of the C open reading frame, the luciferase open reading frame, the HCV 3′ UTR, and a transcription terminator. The helper HCV genomic construct contains a promoter, the complete HCV cDNA, and a terminator.

In one embodiment, the IGVV contains a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme. The IGVV is transfected as DNA into cells expressing the RNA polymerase in the cytoplasm. Such expression is achieved by several methods including cotransfection with a polymerase expression vector, infection with a recombinant vaccinia virus expressing the polymerase, or by previously establishing a cell line permanently expressing the polymerase (see FIG. 3C). The IGVV additionally contains a poly-A or poly-U sequence immediately following the HCV 3′ terminus, so that the transcribed RNA contains a poly-A or poly-U tail at the 3′ end. In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator may be a specific sequence recognized by the bacteriophage RNA polymerase as a termination site or a self-cleaving ribozyme. Alternatively, the terminator is a restriction endonuclease site allowing for linearization of the DNA template prior to transcription (see FIG. 3D). In this case the vector also contains a poly-A or poly-U sequence at the 3′ end.

Resistance Test Vectors—Construction

Resistance test vectors comprising a functional indicator gene expressed from a replication defective minigenome and a helper HCV genomic construct are constructed from the helper HCV genomic construct and patient-derived HCV sequences as described in Example 1. The helper HCV genomic construct is modified to include PSAS for the insertion of NS3/4A or NS5B-containing PDS (described in Example 1, see FIG. 3A). In the case of a drug which targets the function of the IRES, the PSAS are introduced into the minigenome construct as well.

Drug Susceptibility and Resistance Tests

Drug resistance and susceptibility tests using an IGVV system that comprises a functional indicator gene expressed from a minigenome and a helper HCV genomic construct are carried out with resistance test vectors prepared as described above (either as DNA or RNA) by transfection, using either a one cell or two cell assay. Transfection of host cells with the resistance test vectors (the luciferase minigenome plus the helper HCV genomic construct containing the PDS) produces HCV viral particles containing an encapsidated luciferase gene RNA and/or an encapsidated HCV genomic RNA. Drug resistance and susceptibility tests are then performed as described in Example 1.

Drug Screening

Drug screening using an IGVV system that comprises a functional indicator gene expressed from a minigenome and a helper HCV genomic construct is performed essentially as described in Example 1 above.

### EXAMPLE 4

HCV Drug Susceptibility and Resistance Test Using Resistance Test Vectors Comprising Patient-derived Segment(s) and a Nonfunctional Indicator Gene Expressed From Antisense Replication Detective Minigenomes.

Indicator Gene Viral Vector—Construction

Indicator gene viral vectors comprising a non-functional indicator gene expressed from a replication defective minigenome and a helper HCV genomic construct are constructed as follows. The minigenome contains the following elements in a 5′ to 3′ orientation: a promoter sequence, the HCV 3′ UTR (in antisense orientation), the luciferase open reading frame (antisense), the first 24 or 369 nucleotides of the C open reading frame (antisense), the HCV 5′ UTR (antisense), and a transcription terminator. The helper HCV genomic construct contains a promoter, the complete HCV cDNA (in sense orientation), and a terminator.

In this example, the minigenome is introduced into the cells as negative stranded RNA, i.e. as a replicative intermediate RNA copy of the minigenome described above (see FIG. 6). Expression in the transfected cells is dependent on the activity of NS5B, production of which is dependent in turn on the action of NS3/4A and of the cis-acting regulatory elements such as the IRES. Thus the indicator gene is non-functional until acted upon by the viral replication machinery.

In one embodiment, the IGVV contains a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme. The IGVV is transfected as DNA into cells expressing the RNA polymerase in the cytoplasm. Such expression is achieved by several methods including cotransfection with a polymerase expression vector, infection with a recombinant vaccinia virus expressing the polymerase, or by previously establishing a cell line permanently expressing the polymerase (see FIG. 3C). The IGVV additionally contains a poly-A or poly-U sequence immediately following the HCV 3′ terminus, so that the transcribed RNA contains a poly-A or poly-U tail at the 3′ end. In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator may be a specific sequence recognized by the bacteriophage RNA polymerase as a termination site or a self-cleaving ribozyme. Alternatively, the terminator is a restriction endonuclease site allowing for linearization of the DNA template prior to transcription (see FIG. 3D). In this case the vector also contains a poly-A or poly-U sequence at the 3′ end.

Resistance Test Vectors—Construction

Resistance test vectors comprising a non-functional indicator gene expressed from a replication defective minigenome and a helper HCV genomic construct are constructed from the helper HCV genomic construct and patient-derived HCV sequences as described in Example 1. The helper HCV genomic construct is modified to include PSAS for the insertion of NS3/4A or NS5B-containing PDS (described in Example 1, see FIG. 3A). In the case of a drug which targets the function of the IRES, the PSAS are introduced into the minigenome construct as well.

Drug Susceptibility and Resistance Tests

Drug resistance and susceptibility tests using resistance test vectors comprising a non-functional indicator gene expressed from a minigenome and a helper HCV genomic construct are carried out with resistance test vectors prepared as described above (either as DNA or RNA) by transfection, using either a one cell or two cell assay. Transfection of host cells with the resistance test vectors (the luciferase minigenome plus the helper HCV genomic construct containing the PDS) produces HCV viral particles containing an encapsidated luciferase gene RNA and/or an encapsidated HCV genomic RNA. Drug resistance and susceptibility tests are then performed as described in Example 1.

Drug Screening

Drug screening using resistance test vectors comprising a non-functional indicator gene expressed from a minigenome and a helper HCV genomic construct is performed essentially as described in Example 1 above.

### EXAMPLE 5

HCV Drug Susceptibility and Resistance Test Using Resistance Test Vectors Comprising Patient-derived Segment(s) and a Functional Indicator Gene Expressed as a Replication Defective Genome.

Indicator Gene Viral Vector—Construction

Indicator gene viral vectors comprising a functional indicator gene expressed from a replication defective genome and a packaging vector construct are constructed as follows. The IGVV contains the following elements in a 5′ to 3′ orientation: a promoter sequence, the HCV 5′ UTR, the first 24 or 369 nucleotides of the C open reading frame, the indicator gene open reading frame, the NS2 through NS5B portion of the HCV genome (nucleotides 2768-9373 of the H strain of HCV), the HCV 3′ UTR, and a transcription terminator. The packaging vector contains a promoter, the C, E1 and E2 open reading frames of HCV (nucleotides 342-2578 of the H strain of HCV), and a terminator. In the case where the indicator gene is luciferase or another cytoplasmic protein, certain modifications will be made to ensure proper processing of the IG-NS2 junction by the host signal peptidase in the endoplasmic reticulum. In the case of a secreted indicator gene, for example secreted alkaline phosphatase, no such modifications may be required.

Infectious recombinant virions are produced from cells transfected with two vectors: an IGVV containing an IG and the viral non-structural proteins, and a second vector, the packaging vector, containing the viral structural proteins (C/E1/E2; see FIG. 7). To generate infectious particles, the IGVV DNA (or its corresponding RNA, see Example 1, FIGS. 3B-3Alternatively, particles are pseudotyped with envelope glycoprotein genes from related flaviviruses such as BVDV or classical swine fever virus (CSFV). The pseudotyped viruses are used to establish of a cell culture system for single-cycle infection assays. Viruses produced in this manner can then be used to infect target cells, and luciferase expression subsequently measured. This approach has the added advantage of minimizing the amount of manipulations performed with replication competent infectious agents.

In one embodiment, the IGVV contains a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme. The IGVV is transfected as DNA into cells expressing the RNA polymerase in the cytoplasm. Such expression is achieved by several methods including cotransfection with a polymerase expression vector, infection with a recombinant vaccinia virus expressing the polymerase, or by previously establishing a cell line permanently expressing the polymerase (see FIG. 3C). The IGVV additionally contains a poly-A or poly-U sequence immediately following the HCV 31 terminus, so that the transcribed RNA contains a poly-A or poly-U tail at the 3′ end. In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator may be a specific sequence recognized by the bacteriophage RNA polymerase as a termination site or a self-cleaving ribozyme. Alternatively, the terminator is a restriction endonuclease site allowing for linearization of the DNA template prior to transcription (see FIG. 3D). In this case the vector also contains a poly-A or poly-U sequence at the 3′ end.

Resistance Test Vectors—Construction

Resistance test vectors comprising a functional indicator gene expressed from a replication defective genome and a packaging vector construct are constructed from IGVVs described above and patient-derived HCV sequences as described in Example 1. The IGVV is modified to include PSAS for the insertion of NS3/4A, NS5B, or IRES containing PDS (described in Example 1, see FIG. 3A).

Drug Susceptibility and Resistance Tests

Drug resistance and susceptibility tests are carried out with a resistance test vector prepared as described above (either as DNA or RNA) by transfection, using either a one cell or two cell assay. Transfection of host cells with a resistance test vector produces HCV viral particles containing an encapsidated indicator gene RNA. Drug resistance and susceptibility tests are performed as described in Example 1.

Drug Screening

Drug screening using an IGVV comprising a functional indicator gene expressed from a replication defective genome and a packaging vector construct is performed essentially as described in Example 1 above.

### EXAMPLE 6

HCV Protease Inhibitor Susceptibility and Resistance Test Using Resistance Test Vectors Comprising Patient-derived Segment(s) and a Functional Indicator Gene in an NS3/4A BVDV Chimeric Viral Vector.

Indicator Gene Viral Vector—Construction

A chimeric IGVV containing a functional indicator gene and the relevant portion(s) of HCV (for example, the NS3/4A protease domain) were designed with a backbone of a related virus which replicates well in culture. An example of such a virus is BVDV. A complete cDNA for the genome of BVDV has been assembled and shown to generate infectious RNA by in vitrotranscription (Vassilev et al. 1997, J. Virol. 71:471-478). The BVDV polyprotein is processed in a manner very similar to that of HCV, using both host (signal peptidase) and viral encoded proteases. The chimeric IGVV based on a BVDV backbone contains the NS3 protease domain or entire NS3/4A open reading frame of HCV which replaces the corresponding region of BVDV (FIG. 8). By mutating the cleavage sites normally recognized by BVDV NS3 protease to those recognized by HCV NS3/4A, replication of BVDV chimeric RNA and expression of the IG will be dependent on HCV NS3/4A activity.

Chimeric indicator gene viral vectors containing a functional indicator gene in an NS3/4A BVDV chimeric viral vector are constructed as follows. The IGVV contains the following elements in a 5′ to 3′ orientation: a promoter sequence, the BVDV 5′ UTR, the C through NS2 regions of BVDV (NADL strain), the NS3/4A region of HCV, the HCV NS4A/4B cleavage site, the BVDV NS4B open reading frame, the HCV NS4B/SA cleavage site, the BVDV NS5A open reading frame, the HCV NS5A/5B cleavage site, the BVDV NS5B open reading frame, the luciferase open reading frame, the BVDV 3′ UTR and a transcription terminator. In a second embodiment, the IGVV contains the luciferase open reading frame preceeded by an IRES in a similar configuration to that described in Example 2. In a third embodiment, the luciferase gene is expressed from a minigenome similar to that described in Examples 3 or 4.

In one embodiment, the IGVV contains a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme. The IGVV is transfected as DNA into cells expressing the RNA polymerase in the cytoplasm. Such expression is achieved by several methods including cotransfection with a polymerase expression vector, infection with a recombinant vaccinia virus expressing the polymerase, or by previously establishing a cell line permanently expressing the polymerase (see FIG. 3C). The IGVV additionally contains a poly-A or poly-U sequence immediately following the HCV 3′ terminus, so that the transcribed RNA contains a poly-A or poly-U tail at the 3′ end. In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator may be a specific sequence recognized by the bacteriophage RNA polymerase as a termination site or a self-cleaving ribozyme. Alternatively, the terminator is a restriction endonuclease site allowing for linearization of the DNA template prior to transcription (see FIG. 3D). In this case the vector also contains a poly-A or poly-U sequence at the 3′ end.

Resistance Test Vectors—Construction

Resistance test vectors containing a functional indicator gene in an NS3/4A BVDV chimeric viral vector are constructed from IGVVs described above and patient-derived HCV NS3/4A sequences as described in Example 1. The IGVV is modified to include PSAS for the insertion of NS3/4A-containing PDS (described in Example 1, see FIG. 3A).

Drug Susceptibility and Resistance Tests

Drug resistance and susceptibility tests are carried out with a resistance test vector prepared as described above (either as DNA or RNA) by transfection, using either a one cell or two cell assay. Transfection of host cells with a resistance test vector produces HCV viral particles containing an encapsidated indicator gene RNA. Drug resistance and susceptibility tests are performed as described in Example 1.

Drug Screening

Drug screening using an IGVV containing a functional indicator gene expressed from an internal ribosomal initiation sequence is performed essentially as described in Example 1 above.

### EXAMPLE 7

HCV Drug Susceptibility and Resistance Test Using Resistance Test Vectors Comprising Patient-derived Segment(s) and a Functional Indicator Gene in an NS5B BVDV Chimeric Viral Vector.

Indicator Gene Viral Vector—Construction

A chimeric IGVV containing the BVDV structural and non-structural proteins, with the exception of NS5B which is derived from HCV, is designed with a backbone of BVDV. In addition, the BVDV 5′ and 3′ UTRs are replaced with the corresponding regions from HCV, to ensure recognition by the cognate polymerase (FIG. 9).

Indicator gene viral vectors containing a functional indicator gene in an NS5B BVDV chimeric viral vector are constructed as follows. The IGVV contains the following elements in a 5′ to 3′ orientation: a promoter sequence, the HCV 5′ UTR, sequences from the N-terminus of the HCV C open reading frame required for IRES function, the Npro through NS5A regions of BVDV (NADL strain), the NS5B region of HCV, the luciferase open reading frame, the HCV 3′ UTR, and a transcription terminator. In a second embodiment, the IGVV contains the luciferase open reading frame preceeded by an IRES in a similar configuration to that described in Example 2. In a third embodiment, the luciferase gene is expressed from a minigenome similar to that described in Examples 3 or 4.

In one embodiment, the IGVV contains a eukaryotic promoter at the 5′ end of the HCV sequences for the production of RNA in transfected cells, and a transcription terminator at the 31 end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme. The IGVV is transfected as DNA into cells expressing the RNA polymerase in the cytoplasm. Such expression is achieved by several methods including cotransfection with a polymerase expression vector, infection with a recombinant vaccinia virus expressing the polymerase, or by previously establishing a cell line permanently expressing the polymerase (see FIG. 3C). The IGVV additionally contains a poly-A or poly-U sequence immediately following the HCV 3′ terminus, so that the transcribed RNA contains a poly-A or poly-U tail at the 3′ end. In a third embodiment, the IGVV with a bacteriophage RNA polymerase promoter at the 5′ end and a terminator sequence at the 3′ end is transcribed in vitro and the nucleic acid representing the IGVV is transfected as RNA. The terminator may be a specific sequence recognized by the bacteriophage RNA polymerase as a termination site or a self-cleaving ribozyme. Alternatively, the terminator is a restriction endonuclease site allowing for linearization of the DNA template prior to transcription (see FIG. 3D). In this case the vector also contains a poly-A or poly-U sequence at the 3′ end.

Resistance Test Vectors—Construction

Resistance test vectors containing a functional indicator gene in an NS5B BVDV chimeric viral vector are constructed from IGVVs described above and patient-derived HCV NS5B sequences as described in Example 1. The IGVV is modified to include PSAS for the insertion of NS5B-containing PDS (described in Example 1, see FIG. 3A).

Drug Susceptibility and Resistance Tests

Drug resistance and susceptibility tests are carried out with a resistance test vector prepared as described above (either as DNA or RNA) by transfection, using either a one cell or two cell assay. Transfection of host cells with a resistance test vector produces HCV viral particles containing an encapsidated indicator gene RNA. Drug resistance and susceptibility tests are performed as described in Example 1.

Drug Screening

Drug screening using an IGVV containing a functional indicator gene expressed from an internal ribosomal initiation sequence is performed essentially as described in Example 1 above.

### EXAMPLE 8

HCV Drug Susceptibility and Resistance Test Using Resistance Test Vector Systems Comprising Patient-derived Segment(s), A Transcriptional Transactivator, and a Functional Indicator Gene.

Indicator Gene Viral Vector—Construction

An indicator gene viral vector system was designed involving HCV-dependent expression and release of a transcriptional transactivator which activates the expression of an indicator gene. The indicator gene, for example luciferase, is introduced as an expression vector into the host cells by transient or stable transfection. The gene encoding the transactivator protein, for example that of HIV-1, tat, is fused to the HCV polyprotein via a NS3/4A cleavage site linker, in a manner similar to that described for the fusion of luciferase described in Example 1 (i.e. at the C-terminus or elsewhere). Upon expression of the polyprotein, and dependent on the activity of the NS3/4A protease, tat is cleaved from the polyprotein activates the transcription of a reporter gene such as luciferase which is under the control of the HIV-1 long terminal repeat (LTR).

Indicator gene viral vector systems containing a functional indicator gene comprising patient-derived segment (s), a transcriptional transactivator, and a functional indicator gene are constructed as follows. The viral vector contains the following elements in a 5′ to 3′ orientation: a promoter, the HCV 5′ UTR, the open reading frame for the 3010 amino acid HCV polyprotein, containing within it the open reading frame for tat, located variously as described in Example 1, the 3′ UTR, and a transcription terminator. The indicator gene construct contains the HIV-1 LTR, the luciferase open reading frame, and a transcription terminator. The indicator gene construct may be co-transfected with the viral vector, or, preferably, is present as a stable integrated DNA segment in the host cell DNA.

In one embodiment, the viral vector contains a eukaryotic promoter at the 5′ end of the HCV sequences for the production of RNA in transfected cells, and a transcription terminator at the 3′ end. Examples of transcription promoters include, but are not limited to, the CMV intermediate-early promoter, or the SV40 promoter; examples of transcription terminators include, but are not limited to, the transcription terminator/polyadenylation signals found in SV40 or the human β-globin gene (see FIG. 3B). In a second embodiment, the promoter is a promoter for bacteriophage RNA polymerases such as T7, T3, or SP6, and the terminator is a sequence signalling termination of transcription that is recognized by the polymerase, or a self-cleaving ribozyme. The viral vector is transfected as DNA into cells expressing the RNA polymerase in the cytoplasm. Such expression may be achieved by several methods including cotransfection with a polymerase expression vector, infection with a recombinant vaccinia virus expressing the polymerase, or by previously establishing a cell line permanently expressing the polymerase (see FIG. 3C). The viral vector additionally contains a poly-A or poly-U sequence immediately following the HCV 3′ terminus, so that the transcribed RNA contains a poly-A or poly-U tail at the 3′ end. In a third embodiment, the viral vector with a bacteriophage RNA polymerase promoter at the 5′ end and a terminator sequence at the 3′ end is transcribed in vitro and the nucleic acid representing the viral vector is transfected as RNA. The terminator may be a specific sequence recognized by the bacteriophage RNA polymerase as a termination site or a self-cleaving ribozyme. Alternatively, the terminator is a restriction endonuclease site allowing for linearization of the DNA template prior to transcription (see FIG. 3D). In this case the vector also contains a poly-A or poly-U sequence at the 3′ end.

Resistance Test Vectors—Construction

Resistance test vectors containing a functional indicator gene comprising patient-derived segment(s), a transcriptional transactivator, and a functional indicator gene are constructed from viral vectors described above and patient-derived HCV sequences as described in Example 1. The viral vector is modified to include PSAS for the insertion of PDS containing the relevant portion of the HCV genome (described in Example 1, see FIG. 3A).

Drug Susceptibility and Resistance Tests

Drug resistance and susceptibility tests are carried out with a resistance test vector prepared as described above (either as DNA or RNA) by transfection into host cells which contain the indicator construct. Drug resistance and susceptibility tests are performed as described in Example

Drug Screening

Drug screening using indicator gene viral vector systems containing a functional indicator gene comprising patient-derived segment(s), a transcriptional transactivator, and a functional indicator gene is performed essentially as described in Example 1 above.

### EXAMPLE 9

Cytomegalovirus Drug Susceptibility and Resistance Test Using Resistance Test Vectors Comprising Patient-derived Segment (s) and a Functional Indicator Gene Embedded in a Defective Helper Virus.

Indicator Gene Viral Vector—Construction

Indicator gene viral vectors comprising a functional indicator gene inserted into an ORF of HCMV under control of an endogenous viral promoter which in the wild type virus controls the expression of an RNA, for example, the β_{2.7} transcript located in the TR_{L} or “b” repeat (FIG. 12) were designed. The indicator gene viral vector (HCMV-β2.7 F-IG/Δgene X) is further modified and is defective for replication by deleting a segment of the genome containing the viral gene(s) which are the target(s) of the anti-viral drug(s). A viral gene which is the target of an anti-viral drug is refered to herein as gene X. The gene X product is provided on an amplicon plasmid (pA-CMV-VS-gene X) which contains patient sequence acceptor sites (PSAS) and the cis-acting functions required for trans-complementation of the indicator gene viral vector (specifically, these include an HCMV origin of replication and HCMV “a” sequences that direct HCMV genome cleavage and packaging) (FIG. 13). The PSAS are designed to accept the PDS into a cassette containing the regulatory signals appropriate to the individual viral gene/drug target and are derived from the context of the viral gene/drug target in the wild type HCMV. The defective indicator gene viral vector and the amplicon/gene X plasmid constitute a resistance test vector system. The defective indicator gene viral vector is propagated as a viral stock in a packaging host cell/target host cell system in which a functional copy of the viral gene X is provided in trans. Viral stocks from such a packaging host cell/target host cell line are prepared and used to infect cells and/or DNA from these viral stocks is isolated and used to transfect packaging host cell/target host cells as part of a resistance test vector system in conjunction with introduction of the amplicon/gene X plasmid by transfection into a cell type permissive for HCMV infection. Transcomplementation of the deleted gene by the amplicon/gene X plasmid results in a self-perpetuating virus population that results in increased expression of the reporter gene that is dependent on the activity of the viral gene encoded by the patient derived segment that has been introduced into the amplicon/gene X plasmid.

In another embodiment, the amplicon/gene X plasmid (pA-CMV-CS-gene X) contains PSAS that accept the PDS in such a way as to express the patient-derived gene X sequences under control of a heterologous promoter and polyadenylation signals. In one embodiment of this example, an expression cassette containing the CMV IE enhancer promoter region, PSAS, and the SV40 polyadenylation (pA) signal would be included on the amplicon/gene X plasmid (pA-CMV-CS-gene X) in addition to the cis-acting functions required for trans-complementation of the indicator gene viral vector (specifically, these include an HCMV origin of replication and HCMV “a” sequences that direct HCMV genome cleavage and packaging).

In another embodiment the helper viral vector can be supplied as a series of overlapping cosmids that upon transfection into the cell undergo recombination and result in expression of the full array of helper functions. This modification can be used to supply the helper virus sequences in all further examples in the same manner.

In various embodiments of this invention the viral gene/drug target (gene X) can be 1) the HCMV DNA polymerase (UL54), 2) the phosphotransferase (UL97), 3) the viral serine protease (UL80), 4) any viral gene that encodes a real or potential target for a drug susceptibility test or a drug screening test. Such viral gene includes but is not limited to UL44, UL57, UL105, UL102, UL70, UL114, UL98, or UL84.

Plasmids described for the CMV resistance test vectors are named using the following conventions: lower case p indicates that the construction is a plasmid DNA molecule capable of replication in a laboratory strain of E. coli, “A” indicates that the plasmid is an amplicon and thus carries the cis-acting functions required for propagation by a helper virus, specifically these amplicon plasmids contain a viral origin of replication and “a” sequences that direct the genome maturation, cleavage and packaging and make the genomes competent for inversion, CMV indicates that the amplicon sequences are specific for the HCMV (alternatively HSV-1 could indicate the homologous signals from HSV-1 were present on the amplicon), V indicates that the regulatory regions controlling expression of the viral gene/drug target are derived from the HCMV genome and are the regulatory regions used for expression of the viral gene/drug target in the context of the whole virus, C indicates that the regulatory regions used to control expression of the viral gene/drug target are heterologous and in this example comprise the CMV IE promoter/enhancer and the SV40 polyadenylation signal, S indicates that the construct is a sub-genomic construct, gene X identifies the viral gene(s) that is the target(s) of the anti-viral drug(s) and in the examples given here could be UL54, UL80 or UL97 to indicate the DNA polymerase, serine protease, or phosphotransferase, respectively. Helper viruses or indicator gene helper viral vectors are named as follows: HCMV indicates a strain of HCMV, β_{2.7} F-IG indicates a functional indicator gene inserted into the β_{2.7} ORF in the proper reading frame and under control of the β_{2.7} regulatory regions, Δgene X indicates that the viral gene/drug target has been deleted from the virus and in the examples given here could be ΔUL54, ΔUL80 or ΔUL97 to indicate deletion of the DNA polymerase, serine protease, or phosphotransferase, respectively.

Resistance Test Vectors—Construction

Resistance test vectors are prepared by 1) modifying the amplicon/gene X plasmid (pA-CMV-VS-gene X or pA-CMV-CS-gene X) by introducing unique sites, called patient sequence acceptor sites (PSAS) in the gene X coding region, 2) amplifying patient-derived segments (PDS) corresponding to the CMV drug target (gene X) by the amplification of viral DNA present in the blood or tissues of infected patients, and 3) inserting the amplified segments precisely into the amplicon/gene X plasmid at the PSAS. A further embodiment comprises isolation of viral RNA from tissues and using reverse transcription to convert the RNA into DNA copies prior to amplification of the PDS.

Drug Susceptibility and Resistance Tests

Drug susceptibility and resistance tests are carried out with a two part resistance test vector system comprising an amplicon/gene X plasmid (pA-CMV-VS-gene X or pA-CMV-CS-gene X) and corresponding indicator gene viral vector such as HCMV-β_{2.7} F-IG/Δgene X. In one embodiment the amplicon/gene X plasmid is transfected into packaging host cells/target host cells and the cells are then infected with the defective indicator gene viral vector. In another embodiment the amplicon/gene X plasmid and the defective indicator gene viral vector DNA are cotransfected into the packaging host cells/target host cells simultaneously. Packaging host cells/target host cells can be any cells that are permissive for wild type HCMV infection. Transcomplementation of the deleted gene by the amplicon/gene X plasmid results in a self-perpetuating virus population that results in increased expression of the reporter gene that is dependent on the activity of the viral gene/drug target encoded by the patient derived segment that has been introduced into the amplicon/gene X plasmid. Some reporter gene expression will be observed due to the basal level of expression from the defective indicator gene viral vector, however, the replication and thus amplification of the genome of the defective indicator gene viral vector due to the transcomplementation by the amplicon/gene X plasmid will result in a significant increase in the expression of the reporter gene in the target cells. Anti-viral drugs that inhibit HCMV replication through inhibition of the viral gene/drug target will limit the propagation and expansion of the defective indicator gene viral vector, which in turn can be measured as a decrease in the expression of the reporter gene product.

Drug Screening

Drug screening is carried out using a resistance test vector system composed of an amplicon/gene x plasmid (pA-CMV-VS-gene X or pA-CMV-CS-gene X) and an indicator gene viral vector such as HCMV-β_{2.7} F-IG/Δgene X. The PDS may be derived from the genome of a laboratory strain of HCMV or from a patient-derived sample and may be of a wild-type sequence or may contain sequences which render the viral gene/drug target resistant to known anti-viral drugs.

Drug screening is performed as follows: an amplicon/gene X plasmid (pA-CMV-VS-gene X or pA-CMV-CS-gene X) and an indicator gene viral vector such as HCMV-β_{2.7}F-IG/Δgene X are introduced into cells in the absence or presence of potential anti-viral compounds. After maintaining the cultures for an appropriate period of time to allow spread of the defective indicator gene viral vector through the culture, the level of amplicon expression of the reporter gene is measured and the degree of inhibition in the presence of drug is calculated.

### EXAMPLE 10

Cytomegalovirus Drug Susceptibility and Resistance Test Using Resistance Test Vectors Comprising Patient-derived Segment (s) and a Functional Indicator Gene Under Control of a Viral Promoter

Indicator Gene Viral Vector—Construction

A target host cell line is constructed that expresses a functional indicator gene under control of a HCMV viral promoter that is dependent on viral replication for activity. A defective helper viral vector (HCMV/Δgene X) is constructed such that it is defective for replication by virtue of the fact that a segment of the genome containing the viral gene/drug target (gene X) has been deleted from the virus. The viral gene/drug target (gene X) is provided on an amplicon plasmid (pA-CMV-VS-gene X) which contains patient sequence acceptor sites (PSAS) and the cis-acting functions required for trans-complementation of the indicator gene viral vector (specifically, these include an HCMV origin of replication and HCMV “a” sequences that direct HCMV genome cleavage and packaging). The PSAS in pA-CMV-VS-gene X are designed to accept the PDS into a cassette containing the regulatory signals appropriate to the individual viral gene/drug target and are derived from the context of the viral gene/drug target in the wild type HCMV. The defective packaging/helper viral vector (HCMV/Δgene X) and the amplicon/gene X plasmid (pA-CMV-VS-gene X) and the indicator cell line constitute a resistance test vector system. The defective packaging/helper viral vector can be propagated as a viral stock only in a packaging host cell/target host cell system in which the deleted viral gene/drug target is provided in trans. Viral stocks from such a packaging host cell/target host cell line can be prepared and used to infect packaging host cell/target host cells or DNA from these viral stocks can be isolated and used to transfect packaging host cells/target host cells as part of a resistance test vector system in conjunction with introduction of the amplicon/gene X plasmid by transfection into a cell type permissive for HCMV infection. Transcomplementation of the deleted gene by the amplicon/gene X plasmid results in a self-perpetuating virus population that results in increased expression of the reporter gene that is dependent on the activity of the viral gene/drug target encoded by the patient derived segment that has been introduced into the amplicon/gene X plasmid.

In another embodiment, the amplicon/gene X plasmid (pA-CMV-CS-gene X) comprises PSAS that accept the PDS in such a way as to express the patient-derived viral gene drug target (gene X) under control of a heterologous promoter and polyadenylation signals. In one embodiment of this example, an expression cassette comprising the CMV IE enhancer promoter region, PSAS, and the SV40 polyadenylation (pA) signal would be included on the amplicon/gene X plasmid (pA-CMV-CS-gene X) in addition to the cis-acting functions required for trans-complementation of the indicator gene viral vector (specifically, these include an HCMV origin of replication and HCMV “a” sequences that direct HCMV genome cleavage and packaging).

In various embodiments of this invention the viral gene/drug target can be 1) the HCMV DNA polymerase (UL54), 2) the phosphotransferase (UL97), 3) the viral serine protease (UL80), 4) any viral gene that encodes a real or potential target for a drug susceptibility test or a drug screening test. Such viral gene includes but is not limited to UL44, UL57, UL105, UL102, UL70, UL114, UL98, or UL84.

Resistance Test Vectors—Construction

Resistance test vectors are prepared by 1) modifying the amplicon/gene X plasmid (pA-CMV-VS-gene X or pA-CMV-CS-gene X) by introducing unique sites, called patient sequence acceptor sites (PSAS) in the viral gene/drug target (gene X) coding region, 2) amplifying patient-derived segments (PDS) corresponding to the CMV drug target (gene X) from viral DNA present in the blood or tissues of infected patients, and 3) inserting the amplified segments precisely into the amplicon/gene X plasmid at the PSAS. A further embodiment comprises isolation of viral RNA from tissues and using reverse transcription to convert the RNA into DNA copies prior to amplification of the PDS.

Drug Susceptibility and Resistance Tests

Drug susceptibility and resistance tests are carried out with the resistance test vector system comprising an amplicon/gene X plasmid (pA-CMV-VS-geneX or pA-CMV-CS-gene X), a defective viral vector such as HCMV/Δgene X, and a target cell line that contains an indicator gene under the control of an HCMV promoter that is dependent on viral replication for activity. In one embodiment the amplicon/gene X plasmid is transfected into the packaging host cells/target host cells and the cells are then infected with the defective helper viral vector. In another embodiment the amplicon/gene X plasmid and the defective helper viral vector DNA are cotransfected into the packaging host cells/target host cells simultaneously. Transcomplementation of the deleted gene by the amplicon/gene X plasmid results in a self-perpetuating virus population that results in increased expression of the reporter gene that is dependent on the activity of the viral gene/drug target encoded by the patient derived segment that has been introduced into the amplicon/gene X plasmid. Anti-viral drugs that inhibit HCMV replication through inhibition of the viral gene/drug target limit the propagation and expansion of the defective helper viral vector, which in turn is measured as a decrease in the expression of the reporter gene product.

Drug Screening

Drug screening is carried out using a resistance test vector system comprising an amplicon/gene X plasmid (pA-CMV-VS-gene X or pA-CMV-CS-gene X) and a helper viral vector such as HCMV/Δgene X. The PDS may be derived from the genome of a laboratory strain of HCMV or from a patient-derived sample and may be of a wild-type sequence or may contain sequences which render the viral gene/drug target resistant to known anti-viral drugs.

Drug screening is performed as follows: an amplicon/gene X plasmid (pA-CMV-VS-gene X or pA-CMV-CS-gene X) and a helper viral vector such as HCMV/Δgene X are introduced into the indicator cells in the absence or presence of potential anti-viral compounds. After maintaining the cultures for an appropriate period of time to allow spread of the defective helper viral vector through the culture, the level of expression of the reporter gene is measured and the degree of inhibition in the presence of drug is calculated.

### EXAMPLE 11

Cytomegalovirus Drug Susceptibility and Resistance Test Using Resistance Test Vectors Comprising Patient-derived Segment (s) and a Non-Functional Indicator Gene with a Permuted Promoter.

Indicator Gene Viral Vector—Construction

Indicator gene viral vectors comprising a non-functional indicator gene with a permuted promoter are designed using a HCMV amplicon plasmid containing a viral gene which is the target of an anti-viral drug(s) (gene X). The indicator gene viral vector (pA-CMV-VS-gene X-(NF-IG)PP) comprising a non-functional indicator gene with a permuted promoter, all of the cis-acting regulatory elements that are required for HCMV replication (i.e. “a” sequences and the HCMV origin of replication), and a viral gene/drug target expression cassette with PSAS. The PSAS are designed to accept the PDS into a cassette comprising the regulatory signals appropriate to the individual viral gene/drug target and are derived from the context of the viral gene/drug target in the wild type HCMV. The non-functional indicator gene cassette is assembled such that the promoter region is positioned either in the wrong orientation, i.e. anti-sense, with respect to the indicator gene ORF or in the wrong position, i.e. downstream, of the indicator gene ORF (FIG. 14). The promoter and indicator gene ORF are separated and positioned within the regions of the genome that undergo inversion with respect to each other during replication of the genome (FIG. 11). This inversion, when it occurs, brings the two parts of the permuted promoter indicator gene cassette into the proper orientation to allow expression of the indicator gene product. A defective helper viral vector (HCMV/Δgene X) is defective for replication since a segment of the genome containing the viral gene/drug target (gene X) is deleted from the virus. The indicator gene viral vector and the defective helper/packaging viral vector constitute a resistance test vector system. The defective helper viral vector (HCMV/Δgene X) can be propagated only in a cell system in which the deleted viral gene/drug target is provided in trans. Viral stocks packaging host cell/target host cell line can be prepared and used to infect packaging host cells/target host cells or DNA from these viral stocks can be isolated and used to transfect packaging host cells/target host cells as part of a resistance test vector system in conjunction with introduction of the indicator gene viral vector by transfection into a target cell permissive for HCMV infection. Trans-complementation of the deleted gene by the indicator gene viral vector results in a self-perpetuating virus population. During replication of the indicator gene viral vector concatamers of the indicator gene viral vector are formed and inversions occur as part of the normal replication cycle of HCMV (See FIG. 11) and result in a rearrangement of the 2 segments of the permuted promoter cassette such that they now are in the proper orientation to direct transcription of an RNA that will allow expression of the reporter gene.

In another embodiment, the indicator gene viral vector contains PSAS that accept the PDS in such a way as to express the patient-derived gene X sequences under control of a heterologous promoter and polyadenylation signals. In one embodiment of this example, an expression cassette containing the CMV IE enhancer promoter region, PSASs, and the SV40 polyadenylation (pA) signal would be included on the indicator gene viral vector (pA-CMV-CS-gene X-(NF-IG) PP) in addition to the cis-acting functions required for trans-complementation of the indicator gene viral vector (specifically, these comprise an HCMV origin of replication and HCMV “a” sequences that direct HCMV genome cleavage and packaging) and the permuted promoter cassette segments.

In various embodiments of this invention the viral gene/drug target can be 1) the HCMV DNA polymerase (UL54), 2) the phosphotransferase (UL97), 3) the viral serine protease (UL80), 4) any viral gene that encodes a real or potential target for a drug susceptibility test or a drug screening test. Such viral gene includes but is not limited to UL44, UL57, UL105, UL102, UL70, UL114, UL98, or UL84.

Resistance Test Vectors—Construction

Resistance test vectors are prepared by 1) modifying the indicator gene viral vector (pA-CMV-VS-gene X-(NF-IG)PP or pA-CMV-CS-gene X-(NF-IG)PP) by introducing unique restriction sites, called patient sequence acceptor sites (PSAS) in the viral gene/drug target (gene X) coding region, 2) amplifying patient-derived segments (PDS) corresponding to the CMV drug target (gene X) from viral DNA present in the blood or tissues of infected patients, and 3) inserting the amplified segments precisely into the amplicon/gene X plasmid at the PSAS. A further embodiment comprises isolation of viral RNA from tissues and using reverse transcription to convert the RNA into DNA copies prior to amplification of the PDS.

Drug Susceptibility and Resistance Tests

Drug susceptibility and resistance tests are carried out with the resistance test vector system comprising an indicator gene viral vector (pA-CMV-VS-gene X-(NF-IG)PP or pA-CMV-CS-gene X-(NF-IG)PP) and a defective helper viral vector such as HCMV/Δgene X. In one embodiment the indicator gene viral vector (pA-CMV-VS-gene X-(NF-IG)PP or pA-CMV-CS-gene X-(NF-IG)PP) is transfected into appropriate cells and the cells are then infected with the defective helper viral vector (HCMV/Δgene X). In another embodiment the indicator gene viral vector and the defective helper/packaging viral vector DNA are cotransfected into the packaging host cells/target host cells simultaneously. Transcomplementation of the deleted gene by the indicator gene viral vector results in a self-perpetuating virus population. During replication of the indicator gene viral vector concatamers of the indicator gene viral vector are formed and inversions occur as part of the normal replication cycle of HCMV and result in a rearrangement of the 2 segments of the permuted promoter cassette such that they now are in the proper orientation to direct transcription of an RNA that will allow expression of the indicator gene. Expression of the indicator gene is dependent on the activity of the viral gene/drug target encoded by the patient derived segment that has been introduced into the indicator gene viral vector. Anti-viral drugs that inhibit HCMV replication through inhibition of the viral gene/drug target limits the replication of the indicator gene viral vector, which in turn limits the number of genomes in which inversion can occur and can be measured as a decrease in the expression of the reporter gene product.

Drug Screening

Drug screening is carried out using a resistance test vector system comprising an indicator gene viral vector (pA-CMV-VS-gene X-(NF-IG)PP or pA-CMV-CS-gene X-(NF-IG)PP) and a defective packaging/helper viral vector such as HCMV/Δgene X. The PDS may be derived from the genome of a laboratory strain of HCMV or from a patient-derived sample and may be of a wild-type sequence or may contain sequences which render the viral gene/drug target resistant to known anti-viral drugs.

Drug screening is performed as follows: an indicator gene viral vector (pA-CMV-VS-gene X-(NF-IG)PP or pA-CMV-CS-gene X-(NF-IG)PP) and a defective helper viral vector such as HCMV/Δgene X are introduced into cells in the absence or presence of potential anti-viral compounds. After maintaining the cultures for an appropriate period of time to allow replication of the indicator gene viral vector, the level of expression of the reporter gene is measured and the degree of inhibition in the presence of drug is calculated.

### EXAMPLE 12

Cytomegalovirus Drug Susceptibility and Resistance Test Using Resistance Test Vectors Comprising Patient-derived Segment (s) and a Non-Functional Indicator Gene with a Permuted Coding Region.

Indicator Gene Viral Vector—Construction

Indicator gene viral vectors comprising a non-functional indicator gene with a permuted coding region are designed using a HCMV amplicon plasmid containing a viral gene which is the target of an anti-viral drug(s) (gene X). The indicator gene viral vector (pA-CMV-VS-gene X-(NF-IG)PCR) comprises a non-functional indicator gene with a permuted coding region, all of the cis-acting regulatory elements that are required for HCMV replication (i.e. “a” sequences and the HCMV origin of replication), and a viral gene/drug target (gene X) expression cassette with PSAS. The PSAS are designed to accept the PDS into a cassette containing the regulatory signals appropriate to the individual viral gene/drug target and are derived from the context of the viral gene/drug target in the wild type HCMV. The non-functional indicator gene cassette is assembled such that the promoter region and 5′ portion of the coding region are positioned either in the wrong orientation, i.e. anti-sense, with respect to the remaining 3′ portion of the coding region or in the wrong position, i.e. downstream, of the remaining 3′ portion of the coding region (FIG. 15). The promoter and 5′ portion of the coding region are separated from the 3′ portion of the coding region and positioned within the regions of the genome that undergo inversion with respect to each other during replication of the genome (FIG. 11). This inversion, when it occurs, brings the two parts of the permuted coding region indicator gene cassette into the proper orientation to allow expression of the indicator gene product (FIG. 16). A defective helper viral vector (HCMV/Δgene X) is defective for replication by virtue of the fact that a segment of the genome containing the viral gene/drug target (gene X) is deleted from the virus. The indicator gene viral vector and the defective helper/packaging viral vector constitute a resistance test vector system. The defective helper/packaging viral vector (HCMV/Δgene X) is propagated only in a packaging host cell/target host cell system in which the deleted viral gene is provided in trans. Viral stocks from such a packaging host cell/target host cell line can be prepared and used to infect cells or DNA from these viral stocks can be isolated and used to transfect packaging host cells/target host cells as part of a resistance test vector system in conjunction with introduction of the indicator gene viral vector by transfection into a cell type permissive for HCMV infection. Trans-complementation of the deleted gene by the indicator gene viral vector results in a self-perpetuating virus population. During replication of the indicator gene viral vector concatamers of the indicator gene viral vector are formed and inversions occur as part of the normal replication cycle of HCMV and result in a rearrangement of the 2 segments of the permuted coding region cassette such that they now are in the proper orientation to direct transcription of an RNA that will allow expression of the reporter gene (FIG. 16).

In another embodiment, the indicator gene viral vector comprises PSAS that accept the PDS in such a way as to express the patient-derived viral gene sequences (gene X) under control of a heterologous promoter and polyadenylation signals. In one embodiment, an expression cassette comprising the CMV IE enhancer promoter region, PSASs, and the SV40 polyadenylation (pA) signal is included on the indicator gene viral vector (pA-CMV-CS-gene X-(NF-IG)PCR) in addition to the cis-acting functions required for trans-complementation of the indicator gene viral vector (specifically, these include an HCMV origin of replication and HCMV “a” sequences that direct HCMV genome cleavage and packaging) and the permuted coding region cassette segments.

In various embodiments of this invention the viral gene/drug target can be 1) the HCMV DNA polymerase (UL54), 2) the phosphotransferase (UL97), 3) the viral serine protease (UL80), 4) any viral gene that encodes a real or potential target for a drug susceptibility test or a drug screening test. Such viral gene includes but is not limited to UL44, UL57, UL105, UL102, UL70, UL114, UL98, or UL84.

Resistance Test Vectors—Construction

Resistance test vectors are prepared by 1) modifying the indicator gene viral vector (pA-CMV-VS-gene X-(NF-IG)PCR or pA-CMV-CS-gene X-(NF-IG)PCR) by introducing unique restriction sites, called patient sequence acceptor sites (PSAS) in the viral gene/drug target (gene X) coding region, 2) amplifying patient-derived segments (PDS) corresponding to the CMV drug target (gene X) from viral DNA present in the blood or tissues of infected patients, and 3) inserting the amplified segments precisely into the amplicon/gene X plasmid at the PSAS. A further embodiment comprises isolation of viral RNA from tissues and using reverse transcription to convert the RNA into DNA copies prior to amplification of the PDS.

Drug Susceptibility and Resistance Tests

Drug susceptibility and resistance tests are carried out with the resistance test vector system comprising an indicator gene viral vector (pA-CMV-VS-gene X-(NF-IG)PCR or pA-CMV-CS-gene X-(NF-IG)PCR) and a defective helper viral vector such as HCMV/Δgene X. In one embodiment the indicator gene viral vector (pA-CMV-VS-gene X-(NF-IG)PCR or pA-CMV-CS-gene X-(NF-IG)PCR) is transfected into appropriate target host cells and the cells are then infected with the defective helper viral vector (HCMV/Δgene X). In another embodiment the indicator gene viral vector and the defective helper viral vector DNA are co-transfected into the cells simultaneously. Transcomplementation of the deleted gene by the indicator gene viral vector results in a self-perpetuating virus population. During replication of the indicator gene viral vector concatamers of the indicator gene viral vector are formed and inversions occur as part of the normal replication cycle of HCMV and result in a rearrangement of the 2 segments of the permuted coding region cassette such that they now are in the proper orientation to direct transcription of an RNA that will allow expression of the reporter gene (FIG. 16). Expression of the reporter gene is dependent on the activity of the viral gene/drug target encoded by the patient derived segment that has been introduced into the indicator gene viral vector. Anti-viral drugs that inhibit HCMV replication through inhibition of the viral gene/drug target limits the replication of the indicator gene viral vector, which in turn limits the number of genomes in which inversion can occur and can be measured as a decrease in the expression of the reporter gene product.

Drug Screening

Drug screening is carried out using a resistance test vector system comprising an indicator gene viral vector (pA-CMV-VS-gene X-(NF-IG)PCR or pA-CMV-CS-gene X-(NF-IG)PCR) and a defective helper viral vector such as HCMV/Δgene X. The PDS may be derived from the genome of a laboratory strain of HCMV or from a patient-derived sample and may be of a wild-type sequence or may contain sequences which render the viral gene/drug target resistant to known anti-viral drugs.

Drug screening is performed as follows: an indicator gene viral vector (pA-CMV-VS-gene X-(NF-IG)PCR or pA-CMV-CS-gene X-(NF-IG)PCR) and a defective helper viral vector such as HCMV/Δgene X are introduced into cells in the absence or presence of potential anti-viral compounds. After maintaining the cultures for an appropriate period of time to allow replication of the indicator gene viral vector, the level of expression of the reporter gene is measured and the degree of inhibition in the presence of drug is calculated.

### EXAMPLE 13

Cytomegalovirus Drug Susceptibility And Resistance Test Using Resistance Test Vectors Comprising Patient-derived Segment (s) and a Functional Indicator Gene.

Indicator Gene Viral Vector—Construction

Indicator gene viral vectors containing a functional indicator gene under control of an endogenous viral promoter are based on an HCMV amplicon plasmid containing a viral gene which is the target of an anti-viral drug(s) (gene X). The indicator gene viral vector (pA-CMV-VS-gene X-F-IG) comprises a functional indicator gene under the control of a viral promoter dependent on viral replication, all of the cis-acting regulatory elements that are required for HCMV replication (i.e. “a” sequences and the HCMV origin of replication), and a viral gene/drug target (gene x) expression cassette with PSAS (FIG. 17). The PSAS are designed to accept the PDS into a cassette comprising the regulatory signals appropriate to the individual viral gene and are derived from the context of the viral gene/drug target (gene X) in the wild type HCMV. A defective helper viral vector (HCMV/Δgene X) is defective for replication by virtue of the fact that a segment of the genome containing the viral gene/drug target has been deleted from the virus. The indicator gene viral vector and the defective helper viral vector constitute a resistance test vector system. The defective helper viral vector (HCMV/Δgene X) can be propagated only in a packaging host cell/target host cell system in which the deleted viral gene is provided in trans. Viral stocks from such a cell line are prepared and used to infect packaging host cells/target host cells or DNA from these viral stocks are isolated and used to transfect packaging host cells/target host cells as part of a resistance test vector system in conjunction with introduction of the indicator gene viral vector by transfection into a cell type permissive for HCMV infection. Trans-complementation of the deleted gene by the indicator gene viral vector results in a self-perpetuating virus population. Replication of the indicator gene viral vector depends on the transcomplementation between the indicator gene viral vector pA-CMV-VS-gene X-F-IG and the defective helper viral vector HCMV/Δgene X.

In another embodiment, the indicator gene viral vector comprises PSAS that accept the PDS in such a way as to express the patient-derived viral gene/drug target (gene X) under control of a heterologous promoter and polyadenylation signals. In one embodiment, an expression cassette comprising the CMV IE enhancer promoter region, PSAS, and the SV40 polyadenylation (pA) signal is included on the indicator gene viral vector (pA-CMV-CS-gene X-F-IG) in addition to the cis-acting functions required for trans-complementation of the indicator gene viral vector (specifically, these include an HCMV origin of replication and HCMV “a” sequences that direct HCMV genome cleavage and packaging) and the permuted coding region cassette segments.

In various embodiments of this invention the viral gene/drug target can be 1) the HCMV DNA polymerase (UL54), 2) the phosphotransferase (UL97), 3) the viral serine protease (UL80), 4) any viral gene that encodes a real or potential target for a drug susceptibility test or a drug screening test. Such viral gene includes but is not limited to UL44, UL57, UL105, UL102, UL70, UL114, UL98, or UL84.

Resistance Test Vectors—Construction

Resistance test vectors are prepared by 1) modifying the indicator gene viral vector (pA-CMV-VS-gene X-F-IG or pA-CMV-CS-gene X-F-IG) by introducing unique restriction sites, called patient sequence acceptor sites (PSAS) in the viral gene/drug target (gene X) coding region, 2) amplifying patient-derived segments (PDS) corresponding to the CMV drug target (gene X) from viral DNA present in the blood or tissues of infected patients, and 3) inserting the amplified segments precisely into the amplicon/gene X plasmid at the PSAS. A further embodiment comprises isolation of viral RNA from tissues and using reverse transcription to convert the RNA into DNA copies prior to amplification of the PDS.

Drug Susceptibility and Resistance Tests

Drug susceptibility and resistance tests are carried out with the resistance test vector system comprising an indicator gene viral vector (pA-CMV-VS-gene X-F-IG or pA-CMV-CS-gene X-F-IG) and a defective helper viral vector such as HCMV/Δgene X. In one embodiment the indicator gene viral vector (pA-CMV-VS-gene X-F-IG or pA-CMV-CS-gene X-F-IG) is transfected into appropriate packaging host cells/target host cells and the cells are then superinfected with the defective helper viral vector (HCMV/Δgene X). In another embodiment the indicator gene viral vector and the defective helper viral vector DNA are cotransfected into the packaging host cells/target host cells simultaneously. Transcomplementation of the deleted gene by the indicator gene viral vector results in a self-perpetuating virus population that results in increased expression of the reporter gene that is directly dependent on the activity of the viral gene/drug target encoded by the patient derived segment that has been introduced into the indicator gene viral vector. Anti-viral drugs that inhibit HCMV replication through inhibition of the viral gene/drug target will limit the propagation and expansion of the defective helper viral vector, which in turn can be measured as a decrease in the expression of the reporter gene product.

Drug Screening

Drug screening is carried out using a resistance test vector system comprising an indicator gene viral vector (pA-CMV-VS-gene X-F-IG or pA-CMV-CS-gene X-F-IG) and a defective helper viral vector such as HCMV/Δgene X. The PDS may be derived from the genome of a laboratory strain of HCMV or from a patient-derived sample and may be of a wild-type sequence or may contain sequences which render the viral gene/drug target resistant to known anti-viral drugs.

Drug screening is performed as follows: an indicator gene viral vector (pA-CMV-VS-gene X-F-IG or pA-CMV-CS-gene X-F-IG) and a defective helper viral vector such as HCMV/Δgene X are introduced into cells in the absence or presence of potential anti-viral compounds. After maintaining the cultures for an appropriate period of time to allow replication of the indicator gene viral vector, the level of expression of the reporter gene is measured and the degree of inhibition in the presence of drug is calculated.

What is claimed is:

1. A method for determining susceptibility for an HCV anti-viral drug comprising:
(a) introducing a resistance test vector comprising a patient-derived segment which comprises a hepatitis C virus gene and an indicator gene into a host cell;
(b) culturing the host cell from (a);
(c) measuring expression of the indicator gene in a target host cell; and
(d) comparing the expression of the indicator gene from (c) with the expression of the indicator gene measured when steps (a)-(c) are carried out in the absence of the anti-viral drug,
wherein a test concentration of the HCV anti-viral drug is present at steps (a)-(c); at steps (b)-(c); or at step (c).

2. The method of claim 1 wherein the resistance test vector comprises DNA of a genomic viral vector.

3. The method of claim 1 wherein the resistance test vector comprises RNA of a genomic viral vector.

4. The method of claim 1 wherein the resistance test vector comprises genes encoding C, E1, E2, NS2, NS3, NS4, or NS5.

5. The method of claim 1 wherein the patient-derived segment comprises a functional viral sequence.

6. The method of claim 1 wherein the patient-derived segment encodes one protein that is the target of an anti-viral drug.

7. The method of claim 1 wherein the patient-derived segment encodes two or more proteins that are the target of an anti-viral drug.

8. The method of claim 5 wherein the functional viral sequence comprises an IRES.

9. The method of claim 1 wherein the indicator gene is a functional indicator gene and the host cell is a resistance test vector host cell including the additional step of infecting the target host cell with resistance test vector viral particles using filtered supernatants from said resistance test vector host cells.

10. The method of claim 1 wherein the indicator gene is a non-functional indicator gene.

11. The method of claim 10 wherein the host cell is a packaging host cell/resistance test vector host cell.

12. The method of claim 11 wherein the culture is by co-cultivation.

13. The method of claim 11 wherein the target host cell is infected with resistance test vector viral particles using filtered supernatants from said packaging host cell/resistance test vector host cells.

14. The method of claim 1 wherein the indicator gene is a luciferase gene.

15. The method of claim 1 wherein the indicator gene is an β-lactamase gene.

16. The method of claim 11 wherein the packaging host cell/resistance test vector host cell is a human cell.

17. The method of claim 11 wherein the packaging host cell/resistance test vector host cell is a human liver cell.

18. The method of claim 11 wherein the packaging host cell/resistance test vector host cell is a Huh7 cell.

19. The method of claim 1 wherein the target host cell is a HepG2 cell.

20. A resistance test vector comprising a patient-derived segment comprising a gene of Flaviviridae and an indicator gene.

21. The resistance test vector of claim 20, wherein the patient-derived segment comprises a Flavivirus gene.

22. The resistance test vector of claim 20 wherein the patient-derived segment is one gene.

23. The resistance test vector of claim 20 wherein the patient-derived segment is two or more genes.

24. The resistance test vector of claim 20 wherein the patient-derived segment comprises an HCV gene.

25. The resistance test vector of claim 20 wherein the patient-derived segment comprises the NS3/4A protease gene.

26. The resistance test vector of claim 20 wherein the patient-derived segment comprises the NS5B RDRP gene.

27. The resistance test vector of claim 20 wherein the patient-derived segment comprises the IRES.

28. The resistance test vector of claim 20 wherein the indicator gene is a functional indicator gene.

29. The resistance test vector of claim 20 wherein the indicator gene is a non-functional indicator gene.

30. The resistance test vector of claim 20 wherein the indicator gene is a luciferase gene.

31. A packaging host cell transfected with a resistance test vector of claim 20.

32. The packaging host cell of claim 31 that is a mammalian host cell.

33. The packaging host cell of claim 31 that is a human host cell.

34. The packaging host cell of claim 31 that is a human liver cell.

35. The packaging host cell of claim 31 that is HepG2.

36. The packaging host cell of claim 31 that is Huh7.

37. A method for determining susceptibility for an HCV anti-viral drug comprising:
(a) introducing a resistance test vector comprising a patient-derived segment which comprises a hepatitis C virus gene and a nonfunctional indicator gene into a host cell;
(b) culturing the host cell from (a);
(c) measuring expression of the indicator gene in a target host cell; and
(d) comparing the expression of the indicator gene from (c) with the expression of the indicator gene measured when steps (a)-(c) are carried out in the absence of the HCV anti-viral drug,
wherein a test concentration of the HCV anti-viral drug is present at steps (a)-(c); at steps (b)-(c); or at step (c).

38. The method of claim 37 wherein the resistance test vector comprises DNA of a genomic viral vector.

39. The method of claim 37 wherein the resistance test vector comprises RNA of a genomic viral vector.

40. The method of claim 37 wherein the resistance test vector comprises genes encoding C, E1, E2, NS2, NS3, NS4 or NS5.

41. The method of claim 37 wherein the patient-derived segment encodes one protein.

42. The method of claim 37 wherein the patient-derived segment encodes two or more proteins.

43. The method of claim 37 wherein the patient-derived segment comprises a functional viral sequence.

44. The method of claim 37 wherein the indicator gene is a luciferase gene.

45. The method of claim 37 wherein the host cell is a packaging host cell.

46. The method of claim 37 wherein the packaging host cell is a human cell.

47. The method of claim 37 wherein the packaging host cell is a human liver cell.

48. The method of claim 37 wherein the packaging host cell is a Huh7 cell.

49. The method of claim 37 wherein the packaging host cell is a HepG2 cell.

50. The method of claim 37 wherein the nonfunctional indicator gene comprises a negative sense sequence.

51. The method of claim 37 wherein the host cell and target cell are the same cell.

52. The method of claim 37 wherein the target cell is a human cell.

53. The method of claim 37 wherein the target host cell is infected with resistance test vector viral particles using filtered supernatants from said packaging host cell/resistance test vector host cell.

54. The method of claim 37 wherein said culture is by co-cultivation.

55. A method for determining HCV anti-viral drug resistance in a patient comprising:
(a) developing a standard curve of drug susceptibility for an HCV anti-viral drug;
(b) determining HCV anti-viral drug susceptibility in the patient according to the method of claim 1; and
(c) comparing the HCV anti-viral drug susceptibility in step (b) with the standard curve determined in step (a), wherein a decrease in HCV anti-viral susceptibility indicates development of HCV anti-viral drug resistance in the patient.

56. A method for determining HCV anti-viral drug resistance in a patient comprising:
(a) developing a standard curve of drug susceptibility for a HCV anti-viral drug;
(b) determining HCV anti-viral drug susceptibility in the patient according to the method of claim 37; and
(c) comparing the HCV anti-viral drug susceptibility in step (b) with the standard curve determined in step (a), wherein a decrease in HCV anti-viral susceptibility indicates development of HCV anti-viral drug resistance in the patient.

57. A method for determining HCV anti-viral drug resistance in a patient comprising:
(a) determining HCV anti-viral drug susceptibility in the patient at a first time according to the method of claim 1, wherein the patient-derived segment is obtained from the patient at about said time;
(b) determining HCV anti-viral drug susceptibility of the same patient at a later time; and
(c) comparing the HCV anti-viral drug susceptibilities determined in step (a) and (b), wherein a decrease in anti-viral drug susceptibility at the later time compared to the first time indicates development or progression of HCV anti-viral drug resistance in the patient.

58. A method for determining HCV anti-viral drug resistance in a patient comprising:
(a) determining HCV anti-viral drug susceptibility in the patient at a first time according to the method of claim 37, wherein the patient-derived segment is obtained from the patient at about said time;
(b) determining HCV anti-viral drug susceptibility of the same patient at a later time; and
(c) comparing the HCV anti-viral drug susceptibilities determined in steps (a) and (b), wherein a decrease in HCV anti-viral drug susceptibility at the later time compared to the first time indicates development or progression of HCV anti-viral drug resistance in the patient.

59. A method for determining susceptibility for an HCMV anti-viral drug comprising:
(a) introducing a resistance test vector comprising a patient-derived segment which comprises a HCMV gene and an indicator gene into a host cell;
(b) culturing the host cell from (a);
(c) measuring expression of the indicator gene in a target host cell; and
(d) comparing the expression of the indicator gene from (c) with the expression of the indicator gene measured when steps (a)-(c) are carried out in the absence of the anti-viral drug,
wherein a test concentration of the HCMV anti-viral drug is present at steps (a)-(c); at steps (b)-(c); or at step (c).

60. The method of claim 59 wherein the resistance test vector comprises DNA of a genomic viral vector.

61. The method of claim 59 wherein the resistance test vector comprises DNA of a subgenomic viral vector.

62. The method of claim 59 wherein the resistance test vector comprises DNA encoding phosphotransferase (UL 97), DNA polymerase (UL54), protease (UL80), UL54, UL44, UL57, UL105, UL102, UL70, UL114, UL98, or UL84.

63. The method of claim 59 wherein the patient-derived segment comprises a functional viral sequence.

64. The method of claim 59 wherein the patient-derived segment encodes one protein that is the target of an anti-viral drug.

65. The method of claim 59 wherein the patient-derived segment encodes two or more proteins that are the target of an anti-viral drug.

66. The method of claim 59 wherein the indicator gene is a functional indicator gene and the host cell is a resistance test vector host cell including the additional step of infecting the target host cell with resistance test vector viral particles.

67. The method of claim 59 wherein the indicator gene is a non-functional indicator gene.

68. The method of claim 59 wherein the host cell is a packaging host cell/resistance test vector host cell.

69. The method of claim 68 wherein the culture is by co-cultivation.

70. The method of claim 69 wherein the target host cell is infected with resistance test vector viral particles from said packaging host cell/resistance test vector host cells.

71. The method of claim 59 wherein the indicator gene is a luciferase gene.

72. The method of claim 59 wherein the indicator gene is an β-lactamase gene.

73. The method of claim 68 wherein the packaging host cell/resistance test vector host cell is a human cell.

74. The method of claim 68 wherein the packaging host cell/resistance test vector host cell is a human foreskin fibroblast cell.

75. The method of claim 68 wherein the packaging host cell/resistance test vector host cell is a MRC5 cell.

76. The method of claim 59 wherein the target host cell is a human embryonic lung cell.

77. A resistance test vector comprising a patient-derived segment which comprises a gene of herpesviridae and an indicator gene.

78. The resistance test vector of claim 77, wherein the patient-derived segment comprises a alpha herpesvirinae.

79. The resistance test vector of claim 77 wherein the patient-derived segment is one gene.

80. The resistance test vector of claim 77 wherein the patient-derived segment is two or more genes.

81. The resistance test vector of claim 77 wherein the patient-derived segment comprises an HCMV gene.

82. The resistance test vector of claim 77 wherein the indicator gene is a functional indicator gene.

83. The resistance test vector of claim 77 wherein the indicator gene is a non-functional indicator gene.

84. The resistance test vector of claim 77 wherein the indicator gene is a luciferase gene.

85. A packaging host cell transfected with a resistance test vector of claim 77.

86. The packaging host cell of claim 85 that is a mammalian host cell.

87. The packaging host cell of claim 85 that is a human host cell.

88. The packaging host cell of claim 85 that is a human embryonic lung cell.

89. The packaging host cell of claim 85 that is MRC5 cells.

90. The packaging host cell of claim 85 that is a human foreskin fibroblast cell line.

91. A method for determining susceptibility for an HCMV anti-viral drug comprising:
(a) introducing a resistance test vector comprising a patient-derived segment which comprises a HCMV gene and a nonfunctional indicator gene into a host cell;
(b) culturing the host cell from (a);
(c) measuring expression of the indicator gene in a target host cell; and
(d) comparing the expression of the indicator gene from (c) with the expression of the indicator gene measured when steps (a)-(c) are carried out in the absence of the HCMV anti-viral drug,
wherein a test concentration of the HCMV anti-viral drug is present at steps (a)-(c); at steps (b)-(c); or at step (c).

92. The method of claim 91 wherein the resistance test vector comprises DNA of a genomic viral vector.

93. The method of claim 91 wherein the resistance test vector comprises DNA of a subgenomic viral vector.

94. The method of claim 91 wherein the resistance test vector comprises DNA encoding phosphotransferase (UL 97), DNA polymerase (UL54), protease (UL80), UL54, UL44, UL57, UL105, UL102, UL70, UL114, UL98, or UL84.

95. The method of claim 91 wherein the patient-derived segment encodes one protein.

96. The method of claim 91 wherein the patient-derived segment encodes two or more proteins.

97. The method of claim 91 wherein the indicator gene is a luciferase gene.

98. The method of claim 91 wherein the host cell is a packaging host cell.

99. The method of claim 91 wherein the packaging host cell is a human cell.

100. The method of claim 91 wherein the packaging host cell is a human embryonic lung cell.

101. The method of claim 91 wherein the packaging host cell is a human foreskin fibroblast.

102. The method of claim 91 wherein the nonfunctional indicator gene comprises a permuted promoter.

103. The method of claim 91 wherein the nonfunctional indicator gene comprises a permuted coding region.

104. The method of claim 91 wherein the host cell and target cell are the same cell.

105. The method of claim 91 wherein the target cell is a human cell.

106. The method of claim 91 wherein the target host cell is infected with resistance test vector viral particles from said packaging host cell/resistance test vector host cell.

107. The method of claim 106 wherein said culture is by co-cultivation.

108. A method for determining HCMV anti-viral drug resistance in a patient comprising:
(a) developing a standard curve of drug susceptibility for an HCMV anti-viral drug;
(b) determining HCMV anti-viral drug susceptibility in the patient according to the method of claim 59; and
(c) comparing the HCMV anti-viral drug susceptibility in step (b) with the standard curve determined in step (a), wherein a decrease in HCMV anti-viral susceptibility indicates development of HCMV anti-viral drug resistance in the patient.

109. A method for determining HCMV anti-viral drug resistance in a patient comprising:
(a) developing a standard curve of drug susceptibility for an HCMV anti-viral drug;
(b) determining HCMV anti-viral drug susceptibility in the patient according to the method of claim 91; and
(c) comparing the HCMV anti-viral drug susceptibility in step (b) with the standard curve determined in step (a) wherein a decrease in HCMV anti-viral susceptibility indicates development of HCMV anti-viral drug resistance in the patient.

110. A method for determining HCMV anti-viral drug resistance in a patient comprising:
(a) determining HCMV anti-viral drug susceptibility in the patient at a first time according to the method of claim 59, wherein the patient-derived segment is obtained from the patient at about said time;
(b) determining HCMV anti-viral drug susceptibility of the same patient at a later time; and
(c) comparing the HCMV anti-viral drug susceptibilities determined in step (a) and (b), wherein a decrease in anti-viral drug susceptibility at the later time compared to the first time indicates development or progression of anti-viral drug resistance in the patient.

111. A method for determining HCMV anti-viral drug resistance in a patient comprising:
(a) determining HCMV anti-viral drug susceptibility in the patient at a first time according to the method of claim 91, wherein the patient-derived segment is obtained from the patient at about said time;
(b) determining HCMV anti-viral drug susceptibility of the same patient at a later time; and
(c) comparing the HCMV anti-viral drug susceptibilities determined in steps (a) and (b), wherein a decrease in HCMV anti-viral drug susceptibility at the later time compared to the first time indicates development or progression of HCMV anti-viral drug resistance in the patient.
